(19) United States
(12) Patent Application Publication
(10) Pub. No.: US 2023/0096602 A1
(43) Pub. Date: Mar. 30, 2023

(54) TETRAHYDROCYCLOPENTA[B]INDOLE COMPOUNDS FOR TREATMENT OF RENAL DISEASE

(71) Applicant: Eirgen Pharma Ltd., Waterford (IE)

(72) Inventors: Antonio F. CRUZ, Miami, FL (US); Phillip FROST, Miami Beach, FL (US)

(73) Assignee: Eirgen Pharma Ltd., Waterford (IE)

(21) Appl. No.: 17/759,488

(22) PCT Filed: Jan. 26, 2021
(86) PCT No.: PCT/EP2021/051778

§ 371 Date: Jul. 26, 2022

Related U.S. Application Data
(60) Provisional application No. 62/966,382, filed on Jan. 27, 2020.

(51) Int. Cl. A61K 31/4439; A61K 31/593; A61P 13/12; A61K 31/375; A61K 31/355; A61K 31/122; A61K 31/51; A61K 31/714; A61K 31/525; A61K 31/455; A61K 31/197; A61K 31/4188; A61K 31/519

(52) U.S. Cl. CPC A61K 31/4439; A61K 31/593; A61P 13/12; A61K 31/375; A61K 31/355; A61K 31/122; A61K 31/51; A61K 31/714; A61K 31/525; A61K 31/455; A61K 31/197; A61K 31/4188; A61K 31/519

(57) ABSTRACT

Methods of treating renal disease or treating at least one of muscle wasting, low muscle strength, or low physical function in a subject having renal disease by administering at least one tetrahydrocyclopenta[b]indole compound are disclosed. Also disclosed are methods of treating symptoms as a result of secondary hypogonadism induced by renal replacement therapy or kidney failure in a subject having renal disease by administering at least one tetrahydrocyclopenta[b]indole compound. The methods of treatment disclosed herein also include co-administration of the tetrahydrocyclopenta[b]indole compound with a second composition

### FIELD OF THE INVENTION

Methods of treating renal disease or treating at least one of muscle wasting, low muscle strength, or low physical function in a subject having renal disease by administering at least one tetrahydrocyclopenta[b]indole compound are disclosed. Also disclosed are methods of treating symptoms as a result of secondary hypogonadism induced by renal replacement therapy or kidney failure in a subject having renal disease by administering at least one tetrahydrocyclopenta[b]indole compound. The methods of treatment disclosed herein also include co-administration of the tetrahydrocyclopenta[b]indole compound with a second composition.

### BACKGROUND OF THE INVENTION

Nearly 750,000 people are treated for end-stage renal disease (ESRD) in the US alone, and approximately 500,000 are dialysis-dependent (National Kidney Foundation, NIH, CDC, accessed October 2019). The prevalence of ESRD is slightly higher in men vs women (CDC, 2019). The number of ESRD patients is estimated to increase to approximately 1,250,000 in 2030 due to increasing rates of diabetes and obesity and changes in race distribution (McCullough, Keith P., et al. “Projecting ESRD incidence and prevalence in the United States through 2030.” Journal of the American Society of Nephrology 30.1(2019): 127-135). Current worldwide estimate of ESRD is 2 million patients with increasing rates of 5-7% annually (Centers for Disease Control and Prevention, “Chronic Kidney Disease in the United States, 2019, accessed https://www.cdc.gov/kidneydisease/publications-resources/2019-national-facts.html, October 2019), and this number is most likely an underestimate in low economy countries due to lack of access to healthcare.

Frailty, an important, multifactorial component of ESRD is defined as the state of low homeostatic built-in reserve, leading to a high vulnerability for dramatic sudden changes in health due to advancing age and chronic disease. ESRD is associated with increased frailty, especially in patients >50-80+ years old (66.4-78.8%) and women (Abdel-Rahman, Emaad, “Association Between CKD and Frailty and Prevention of Functional Losses.” Geriatric Nephrology Curriculum (2009; Drost, Diederik, et al. “High prevalence of frailty in end-stage renal disease.” International urology and nephrology 48.8 (2016): 1357-1362). Frail patients on hemodialysis are at higher risk for death (2.6×) and hospitalization (1.4×) than non-frail patients (McAdams-DeMarco, Mara A., et al. “Frailty as a novel predictor of mortality and hospitalization in individuals of all ages undergoing hemodialysis.” Journal of the American Geriatrics Society 61.6 (2013): 896-901). Frailty in ESRD includes sarcopenia, defined as muscle failure with low muscle strength and an acute or chronic loss of muscle (Cruz-Jentoft, Alfonso J., et al. “Sarcopenia: revised European consensus on definition and diagnosis.” Age and ageing 48.1 (2018): 16-31; Vellas, Bruno, et al “Implications of ICD-10 for sarcopenia clinical practice and clinical trials: report by the International Conference on Frailty and Sarcopenia Research Task Force.” The Journal of frailty & aging 7.1 (2018): 2-9; Abdel-Rahman 2009 ibid.). Sarcopenia in ESRD patients is a common problem with rates up to 63% (Bae, Eun Hui. “Is sarcopenia a real risk factor for mortality in patients undergoing hemodialysis?.” The Korean journal of internal medicine 34.3 (2019): 507). Sarcopenia is strictly correlated with physical disability, poor quality of life and death (Santilli, Valter, et al. “Clinical definition of sarcopenia.” Clinical cases in mineral and bone metabolism 11.3 (2014): 177), and increased likelihood of falls and fractures (Cruz-Jentoft et al., 2019). Development of sarcopenia may be associated with conditions that are not exclusively present in older population (Cruz-Jentoft et al., 2019; Santilli et al., 2014). For example, hospitalization/nursing-home residency alone lead to acute sarcopenia rates up to 32.8% in non-ESRD patients (Welch, Carly, et al. “Acute sarcopenia secondary to hospitalisation-an emerging condition affecting older adults.” Aging and disease 9.1 (2018): 151).

Androgen deficiency is another common cause of sarcopenia, frailty and mortality. Androgens exert a direct effect on the human skeletal muscle strength via induction of muscle hypertrophy (Herbst, Karen L., and Shalender Bhasin. “Testosterone action on skeletal muscle.” Current Opinion in Clinical Nutrition & Metabolic Care 7.3 (2004): 271-277). A total of 6-12% men in general population suffer from low testosterone, and up to 77% of ESRD patients have decreased levels of testosterone (≤420 ng/dL [14 nmol/L]) (Snyder, Grace, and Daniel A. Shoskes. “Hypogonadism and testosterone replacement therapy in end-stage renal disease (ESRD) and transplant patients.” Translational andrology and urology 5.6 (2016): 885). A decrease of 2.18 standard deviation (SD) in total testosterone in general population has been associated with a 35% increased risk of all-cause mortality (Araujo, Andre B., et al. “Endogenous testosterone and mortality in men: a systematic review and meta-analysis.” The Journal of Clinical Endocrinology & Metabolism 96.10 (2011): 3007-3019; Garibotto, Giacomo, Daniela Picciotto, and Daniela Verzola. “Testosterone deficiency, frailty and muscle wasting in CKD: a converging paradigm?.” (2018): 723-726). Recently, low levels of serum testosterone (<260-270 ng/dL [<9 nmol/L]) have been shown to be associated with infection-related hospitalization and all-cause mortality in hemodialysis male patients (Nakashima, Akio, et al. “Associations between low serum testosterone and all-cause mortality and infection-related hospitalization in male hemodialysis patients: a prospective cohort study.” Kidney international reports 2.6 (2017): 1160-1168). Finally, a 50% lower concentration of free testosterone was associated with higher likelihood of becoming frail within 12 month as measured by reduced grip strength and gait speed in ESRD patients, and developing sarcopenia as inferred from lower muscle mass at 12 months (Chiang, Janet M., et al. “Low testosterone is associated with frailty, muscle wasting and physical dysfunction among men receiving hemodialysis: a longitudinal analysis.” Nephrology Dialysis Transplantation 34.5 (2018): 802-810).

The prevalence of hypogonadism associated with renal failure is escalating and is associated with significant morbidity which includes premature cardiovascular disease and anemia (Thirumavalavan et al., Indian J Urol 2015; 31(2): 89-93). Other conditions associated with hypogonadism include obesity, osteoporosis, HIV, and COPD. Patients having renal failure also often have co-morbid conditions such as hypertension, cardiovascular disease and obesity. Normal testosterone values of greater than 14 nmol/L are found in only about 23% of patients with ESRD. A total testosterone level of less than 10 nmol/L (300 ng/dL) is often used as the cut-off level for low testosterone—i.e., testosterone deficiency. Testosterone insufficiency is typically defined as between 10-14 nmol/L.

Patients on dialysis may also suffer even further drops in testosterone levels, making this patient population particularly vulnerable to morbidity associated with renal failure and dialysis. Some drugs commonly prescribed to ESRD patients directly inhibit sex hormone synthesis. These medications include spironolactone and cimetidine which compete for androgen receptors while also inhibiting 17-alpha hydroxylase and C17-C20 lyase activity which reduces testosterone synthesis. Glucocorticoids and immunosupressants also decrease T synthesis. Secondary hypogonadism may also be induced by various drugs such as antidepressants, benzodiazepines and opiates which inhibit LH and FSH signaling. (Schmidt et al. Sexual hormone abnormalities in male patients with renal failure. Nephrol Dial Transplant. 2002; 17:368-71).

CKD patients also show decreasing levels of total and free testosterone as the CKD stages go from stage 1 to stage 5 (Yilmaz et al., 2011). In the Yilmaz study, 17% of the CKD stage 1 patients had low or lower levels of total and free T while 57% of stage 5 CKD patients had such low or lower levels.

Hypogonadism in male patients with renal failure has been managed with various testosterone replacement options-all of which have risks associated with the administration of this hormone. Supratherapeutic peaks from intramuscular injections can lead to polycythemia which is a serious risk for ESRD patients having cardiovascular disease. Subcutaneous implants of T in pellets can lead to pellet extrusion, minor bleeding and other conditions such as infection or fibrosis.

Testosterone replacement therapy itself may also cause various side effects such as weight gain, edema, gynecomastia and/or polycythemia.

Thus, it is believed that the present invention meets a serious unmet medical need in a particular vulnerable ESRD patient population by providing a selective androgen receptor modulator (SARM) which has the positive attributes of testosterone replacement therapy including improvement in lean muscle mass while simultaneously avoiding the negative symptoms associated with such therapy—e.g. increase in prostate size, etc.

In summary, frailty and sarcopenia both share common factors including diminished physical function, decreased muscle mass, lower muscle strength, and are important markers of mortality, hospitalization and poor quality of life in the ESRD patient population.

Kidney dialysis patients experience accelerated protein catabolism leading to muscle wasting, a marker for increased mortality vs non-dialysis patients (Chen, Chun-Ting, et al. “Muscle wasting in hemodialysis patients: new therapeutic strategies for resolving an old problem.” The Scientific World Journal 2013 (2013). Recent evidence suggests that assessment of muscle strength (i.e. function) is a better predictor of outcome and comorbidities than muscle mass (Stenvinkel, Peter, et al. “Muscle wasting in end-stage renal disease promulgates premature death: established, emerging and potential novel treatment strategies.” Nephrology Dialysis Transplantation 31.7 (2015): 1070-1077). The current international criteria for diagnosis of sarcopenia include the measurements of lean mass and mobility/performance criteria via gait speed, 6-minute walk test (6-MWT), and grip strength (Rooks, Daniel, and Ronenn Roubenoff. “Development of Pharmacotherapies for the Treatment of Sarcopenia.” The Journal of Frailty & Aging (2019): 1-11). Belgian Society of Gerontology and Geriatrics has recently published a recommendation for testosterone supplementation. A possible testosterone intervention has been recommended to improve muscle mass and muscle strength in male patients with sarcopenia >65 years old with low serum testosterone levels (<200-300 ng/dL) and clinical muscle weakness. The side effect target monitoring of hematocrit, lipid profile and prostate specific antigen (PSA), for testosterone therapy has been included in this guidance (De Spiegeleer, Anton, et al. “Pharmacological interventions to improve muscle mass, muscle strength and physical performance in older people: an umbrella review of systematic reviews and meta-analyses.” Drugs & aging 35.8 (2018): 719-734). The conclusive efficacy of testosterone replacement on muscle mass and strength has not yet been demonstrated in chronic kidney disease (CKD) and dialysis patient populations (Snyder and Shoskes, 2016).

In healthy females, the most significant testosterone decreases are noted between the ages 21-40 with a 50% decrease (approx. 20 ng/dL) by 40 years of age (Zumoff, Barnett, et al. “Twenty-four-hour mean plasma testosterone concentration declines with age in normal premenopausal women.” The Journal of Clinical Endocrinology & Metabolism 80.4 (1995): 1429-1430)). Physical frailty in older females is 2 times more prevalent than in males (Ruan, Qingwei, et al. “Sexual dimorphism of frailty and cognitive impairment: Potential underlying mechanisms.” Molecular medicine reports 16.3 (2017): 3023-3033). Recent pooled data analysis of 8 clinical trials (N=500) by Islam and colleagues showed no effect of testosterone on muscle strength, body composition and bone parameters. Moreover, data in that report showed increased occurrence of acne and hair growth in testosterone-treated group vs placebo across 11 studies (N=3000-4000). No serious adverse events or cardiac events were more frequent with testosterone treatment (Islam, Rakibul M., et al. “Safety and efficacy of testosterone for women: a systematic review and meta-analysis of randomised controlled trial data.” The Lancet Diabetes & Endocrinology 7.10 (2019): 754-766). However, testosterone replacement therapy has been suggested to delay the progression of CKD and ESRD (Goel et al. “Effects of Online Educational Interventions in Hyperkalemia Nephrologist. Spring Clinical Meetings (Abstract), 2017). Therefore, more tissue-specific factors including selective androgen receptor modulators (SARMs) could potentially alleviate symptoms associated with ESRD in female patients without the virilizing adverse effects accompanying steroidal androgens.

Selective androgen receptor modulators (SARMs) act as tissue-specific agents with antagonistic effects on the prostate and anabolic effects on body composition, muscles and bone, crucial for the management of frail ESRD patients suffering from sarcopenia. A large body of data are in agreement that SARMs improve muscle mass and strength. Finally, SARMS are potentially free of side effects of testosterone and anabolically more efficacious due its specificity (Bhasin, Shalender, et al. “Drug insight: testosterone and selective androgen receptor modulators as anabolic therapies for chronic illness and aging.” Nature Reviews Endocrinology 2.3 (2006): 146). The proposed mechanisms for the tissue selectivity of SARMs include their inability to serve as substrates for steroid 5α-reductase enzymes (SRD5A1 and SRD5A2), differences in the conformations acquired by the AR after binding to SARM molecule versus testosterone, and the recruitment of a unique repertoire of tissue-specific co-regulators, resulting in activation of tissue-specific intracellular signaling cascades and gene transcription (Narayanan, Ramesh, et al. “Selective androgen receptor modulators in preclinical and clinical development.” Nuclear receptor signaling 6.1 (2008): nrs-06010). Disclosed herein are oral SARMs with tissue selectivity to the human androgen receptor (hAR), previously demonstrated in vitro. The SARMs disclosed herein ameliorate and potentially prevent the symptoms of muscle wasting and metabolic abnormalities in ESRD patients.

SARMS, such as compound of Formula I or Formula II, are orally bioavailable and tissue-selective, whereas testosterone and other anabolic steroids also have limited oral bioavailability and are only available in transdermal and intramuscular formulations potentially leading to skin reactions and fluctuations in serum concentrations of testosterone. SARMS may exhibit the beneficial effects of anabolic agents without the known associated risks (Mohler M L, Bohl C E, Jones A, et al. Nonsteroidal selective androgen receptor modulators (SARMs): Dissociating the anabolic and androgenic activities of the androgen receptor for therapeutic benefit. J Med Chem 2009, 52(12): 3597-3617).

The compounds disclosed in U.S. Pat. No. 7,968,587 are useful in the treatment of disorders typically treated with androgen therapy. These disorders include hypogonadism, reduced bond mass or density, osteoporosis, osteopenia, reduced muscle mass, strength and function, sarcopenia, age related functional decline, delayed puberty in boys, anemia, male or female sexual dysfunction, erectile dysfunction, reduced libido, depression, and lethargy. The compounds are described as potent androgen receptor (AR) ligands that agonize the androgen receptor and selectively bind thereto (SARMs). However, the patent does not disclose the use of these compounds to treat End Stage Renal Disease (“ESRD”) or Chronic Kidney Disease (“CKD”). In addition, there is no disclosure therein that such compounds are potent antagonists on the prostate at low doses and lack agonist activity on the prostate even at very high doses. WO 2016040234 discloses the use of (S)-(7-cyano-4-pyridin-2-ylmethyl-1,2,3,4-tetrahydro-cyclopenta[b]indol-2-yl)-carbamic acid isopropyl ester (TT701) to treat androgen deprivation therapy associated symptoms. Data was presented therein from various animals including rats and dogs that showed treatment with TT701, at the doses provided, for a period of 1 to 12 months, decreased prostate size in the rats and dogs which indicated that the compound does not accrue androgenic risk of prostate hyperplasia over time. The treatment of TT701 in dogs for 6 and 12 months resulted in a 60% to 80% decrease in prostate weight and the presence of atrophy. This data alone, or the other safety or clinical data disclosed therein, is not dispositive of the treatment of any indication in humans except for the treatment of androgen deprivation symptoms. The data shown therein also disclosed that there were no significant changes from baseline in prostate specific antigen (PSA) levels when compared with placebo at any time point or any dose tested of TT701 in healthy volunteers. The present invention broadly relates to the discovery that a compound of formula I, inclusive of TT701, and, optionally, a combination with a vitamin D pro-hormone such as 25hydroxyvitamin D (D2 or D3) or controlled release formulations thereof or other vitamin D active hormones or analogs thereof is (are) useful for the treatment of the signs and symptoms of ESRD and/or CKD in patients in need of treatment thereof. The term “patient” or “patients” is inclusive of humans and animals.

There exists, in the art, a need for a SARM that can effectively treat the distressing symptoms of ESRD and improve body composition changes would be an innovative clinically beneficial therapeutic option for frail ESRD patients with sarcopenia. As the ESRD rates are continuously increasing every year there is a great need for development of suitable supportive therapies to improve physical function and quality of life of this vulnerable population.

### SUMMARY OF THE DISCLOSURE

In one embodiment, the invention relates to a method of treating symptoms or conditions associated with renal disease in a subject in need of treatment thereof comprising administering a therapeutically effect amount of at least one tetrahydrocyclopenta[b]indole compound to said subject, wherein the tetrahydrocyclopenta[b]indole compound has Formula I:

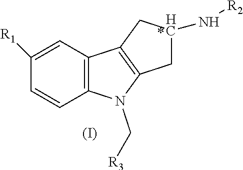

wherein the C* atom may be R, S or R/S configuration;
R1 represents cyano, —CH═NOCH3, —OCHF2, or —OCF3;
R2 represents —COR2a or —SO2R2b;
R2a represents (C1-C4)alkyl, (C1-C4)alkoxy, cyclopropyl, or —NRaRb;
R2b represents (C1-C4)alkyl, cyclopropyl, or —NRaRb;
Ra and Rb each independently is H or (C1-C4)alkyl; and
R3 represents a heteroaryl group selected from the group consisting of pyridinyl, pyrimidinyl, pyrazinyl, pyridazinyl, thiazoloy isothiazolyl, and thiadiazolyl, each optionally substituted with 1 or 2 substituents independently selected from the group consisting of methyl, ethyl, bromo, chloro, fluoro, —CHF2, —CF3, hydroxyl, amino and —NHCH2CO2H, or a pharmaceutically acceptable salt thereof. In one embodiment the subject being treated has stage 3, 4 or 5 chronic kidney disease or secondary hypogonadism induced by renal replacement therapy or kidney failure in a subject having renal disease. In one embodiment, the tetrahydrocyclopenta[b]indole compound is carbamic acid, N-[(2S)-7-cyano-1,2,3,4-tetrahydro-4-(2-pyridinylmethyl)cyclopent[b]indol-2-yl]-, 1-methylethyl ester or has the structure of formula II:

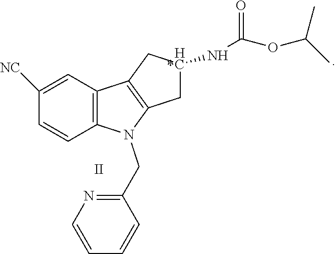

In one embodiment, the invention relates to (i) a method of treating end stage renal disease or treating at least one of muscle wasting, low muscle strength, or low physical function in a subject having end stage renal disease or (ii) symptoms as a result of secondary hypogonadism induced by renal replacement therapy or kidney failure in a subject having end stage renal disease, comprising administering a therapeutically effect amount of the compound of Formula II or a pharmaceutically acceptable salt thereof to said subject, wherein the compound has Formula II:

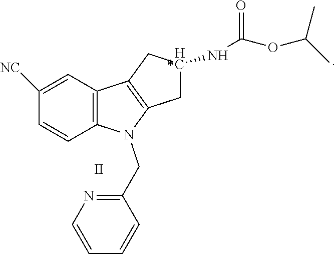

In one embodiment, the subject being treated has chronic kidney disease (“CKD”), stage five CKD, end-stage renal disease (“ESRD”), and/or is undergoing dialysis.

In one embodiment, the invention relates to the methods of treatment previously described and further comprising administering a therapeutically effect amount of a second composition (“second compound”) to said subject. In one embodiment, the second compound administered is carotenoids, vitamin C, vitamin D, vitamin E, vitamin K, vitamin B1, vitamin B2, vitamin B3, vitamin B5, vitamin B6, vitamin B7, vitamin B9, vitamin B12, or a vitamin D pro-hormone. In one embodiment, the second compound administered is extended-release calcifediol (ERC). In one embodiment, the ERC is (3β,5Z,7E)-9,10-secocholesta-5,7,10(19)-triene-3,25-diol monohydrate. I one embodiment the second compound administered is calcitriol or a vitamin D analog.

In one embodiment, the invention relates to a pharmaceutical composition comprising a compound of formula I or formula II and an extended release dosage form of calcifediol and acceptable excipients in a fixed unit combination oral dosage form. In one embodiment, the formula II is carbamic acid, N-[(2S)-7-cyano-1,2,3,4-tetrahydro-4-(2-pyridinylmethyl)cyclopent[b]indol-2-yl]-, 1-methylethyl.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE FIGURES

FIG. 1 illustrates the results that daily oral administration of the compound of Formula II led to a dose-dependent increase in vertebral bone mineral content (BMC), cross-sectional area, and bone mineral density (BMD).

FIG. 2 illustrates the results of one way analysis of levator ani W/BW (mg/g) in delayed rat ORX model.

FIG. 3 illustrates the results of the compound of Formula II treatment in the ORX rat that showed minimal accrual SV/Prostate risk

FIG. 4 illustrates the effect of Testosterone Enanthate (TE) and the compound of Formula II on Prostate weight.

FIG. 5 illustrates the effect of Testosterone Enanthate (TE) and the compound of Formula II on Seminal Vesicle weight.

FIG. 6 illustrates the results of the compound of Formula II Phase I PSA results in healthy volunteers (ug/L).

FIG. 7 illustrates the PSA mean change from baseline by treatment and visit day in the healthy volunteers: Study GPEC (nanogram/mL).

FIGS. 8 and 9 show the changes in PSA levels (ng/ml) in patients having symptoms of erectile dysfunction and who were failing tadalafil after treatment using OPK-88004 (TT701) alone or in combination with tadalafil (5 mgs and 5 mgs). FIG. 8 also shows the data with respect to tadalafil alone.

FIG. 10 shows the changes in PSA levels in patients having symptoms of erectile dysfunction and who were failing tadalafil treatment being administered OPK-88004 (TT701) alone or in combination with tadalafil (5 mgs alone or 5 mg/5 mg combination) where the PSA horizontal scale is broadened. This figure shows a decrease in PSA levels at about 2.0 ng ml upon treatment with OPK-88004.

FIG. 11 illustrates that the compound of Formula II causes no change in Hematocrit with TT701 in 12 weeks.

FIG. 12 illustrates the LBM mean change from baseline to week 12: Study GPEC.

FIG. 13 illustrates the Muscle Power (stair climb) mean change from baseline to week 12: Study GPEC.

FIG. 14 illustrates the fat Mass mean change from baseline to week 12: Study GPEC.

FIG. 15 shows the study design outlined in Example 15.

FIG. 16 shows the changes in PSA levels in patients being administered tadalafil alone (5 mg and 10 mg) or being administered OPK-88004 (TT701) (1 mg or 5 mg) in combination with tadalafil (5 mg) after 12 weeks of treatment.

FIG. 17 shows a simulation of concentrations profile of OPK-88004 for once daily administration for 14 days.

FIG. 18 shows the simulated PK characteristics between the GPBC study and the GPEC study.

### DETAILED DESCRIPTION OF THE INVENTION

The invention encompasses methods of treating renal disease, including CKD and ESRD, by administering at least a compound of Formula I in a therapeutically effective amount to a subject in need of treatment thereof. Also, the invention encompasses treating renal disease by administering a pharmaceutical composition comprising at least one compound of Formula I or a pharmaceutically acceptable salt thereof and a pharmaceutically acceptable excipient and dosage forms thereof. The compounds of Formula I may be prepared by the methods described in U.S. Pat. No. 7,968,587, hereby incorporated by reference or as described in WO 2016/040234 A1 (PCTUS2015/048801) which is hereby incorporated by reference.

The inventors found that compounds for Formula I and, in particular, a compound of Formula II (described below, also known as LY2452473, TT701, OPK-88004 or (S)-(7-cyano-4-pyridin-2-ylmethyl-1,2,3,4-tetrahydro-cyclopenta[b]incol-2-yl)-carbamic acid isopropyl ester), are potent and selective modulators of the human androgen receptor (hAR) in cell based assays.

The SARM compounds of Formula I and Formula II can ameliorate and potentially prevent the symptoms of muscle wasting and metabolic abnormalities in ESRD patients. In vitro and animal data support that the SARM compounds of Formula I and Formula II serve as a potential antagonist to testosterone on the prostate and at similar doses provides anabolic activity on muscle and bone. In a gonadectomized rodent model, SARM compounds of Formula I and Formula II demonstrated anabolic effects on muscle and osteoanabolic properties on bone mass and biomechanical strength. In a multiple dose trial, healthy subjects exposed for 28 days to SARM compounds of Formula II demonstrated clinically and statistically significant increases in LBM and calf area. This was accompanied by changes in bone biochemical biomarkers consistent with a bone anabolic increase (data not shown). In a phase 2 trial in men with ED, SARM compounds of Formula II has shown improvement on body composition parameters including lower extremity muscle strength and power, LBM and fat mass after 12 weeks of treatment.

As used herein the term “(C₁-C₄)alkyl” means a straight or branched, monovalent, saturated aliphatic chain of one to four carbon atoms.

As used herein, the term (C₁-C₄)alkoxy means an oxygen atom bearing a straight or branched alkyl chain as described above.

As used herein, the terms “halo,” “halide,” or “Hal” refer to chlorine, bromine, iodine or fluorine unless stated otherwise.

As used herein, the term “patient” includes mammals such as humans, dogs, cats, cows, horse, pigs, or sheep or other mammal.

As used herein, the term “treating” or “treatment” means administering at least one drug or a combination thereof to alleviate and treat the underlying signs, causes or symptoms of a disease or condition. This term includes any form of prohibiting, slowing, stopping or otherwise interfering with disease progression. The preferred mammal to treat is humans and the indication being treated is treating end stage renal disease (e.g. patients on dialysis or CKD stage 5) or treating at least one of muscle wasting, low muscle strength, or low physical function in a subject having renal disease. The preferred patient population is adults older than 50 years. Patients who are pre-dialysis or having stage 3 or 4 CKD or ESRD may also receive treatment with a compound of formula I or II or combinations of such compounds with an extended release formulation of a vitamin D pro-hormone such as 25(OH)D3 or 25(OH)D2 or other therapy such as calcitriol or a vitamin D analog.

As used herein, the term “effective amount” refers to the amount or does of compound of Formula I or Formula II, or a pharmaceutically acceptable salt thereof, upon administration to the patient, provides the desired effect in the patient under diagnosis or treatment. In determining the effective amount for a patient, a number of factors are considered by the attending diagnostician including, but not limited to, the patient's size, age, and general health; the specific disease or disorder; the response of the individual patient; the particular compound administered; the mode of administration; the bioavailability characteristics of medication; and other relevant circumstances.

Animal studies determined that the compound of Formula II (TT701, OPK-88004, or OPK88004) showed selectivity for the anabolic effects relative to the prostate androgenic effects. The ED₅₀ for the levator ani muscle was 1-3 mg/kg whereas doses of 30 mg/kg, the highest dose examined, did not induce changes in the prostate of orchidectomized rats. This result suggests at least a 10-30 fold selectivity.

The treatment of normal dogs with the compound of Formula II yielded a progressive decrease in prostate size by 60% over a six-month treatment period. Similar antagonist effects on prostate weight were observed with treatment doses of 3, 30 and 300 mgs/kg, whereas increase in anabolic activity was observed in skeletal muscle and bone. These data support that compounds of Formula I and, in particular, Formula II work as antagonists to endogenous androgenic related effects on the prostate.

Orchidectomized rats with reduced prostate weights were treated with testosterone alone or with testosterone and the compound of Formula II. Testosterone treatment alone only partially reversed the effect, however it also increased prostate weight. In contrast, the combination of testosterone and the compound of Formula II reduced testosterone induced effects on prostate size, indicating that the compound of Formula II may act as an androgen antagonist on the prostate.

In clinical studies in patients with androgen deficiency, treatment with TT701, the compound of Formula II, resulted in a 20-30% decrease in the levels of endogenous testosterone. The exact effect of this decrease in testosterone levels on androgen related anabolic and prostate effects is not known, but may be dependent on the base levels of testosterone.

In these same clinical studies in patients with androgen deficiency, TT701 demonstrated clinically and statistically significant increases in lean body mass and changes in bone biochemical biomarkers consistent with a bone anabolic increase. No increases in PSA were observed at any dose level (up to 75 mg doses) indicating that the compound of Formula II acts as a selective AR modulator in humans (agonist effects on some tissues, neutral or antagonistic effect on the prostate), supporting the data generated in animal models.

In clinical studies for the potential treatment of patients with androgen deficiency, TT701 showed a good safety profile within the dose ranges studied. The major changes observed in patients treated with 5 mg of the compound of Formula II for 12 weeks was a 20% decrease in HDL, and some decrease in sex hormone binding globulin, LH and FSH, but the magnitude of these findings were not considered clinically relevant.

The present invention also relates to use of TT701 and compounds of formula I in the treating renal disease or treating at least one of muscle wasting, low muscle strength, or low physical function in a subject having renal disease. The preferred patient population is patients, male or female, having ESRD.

The compound of Formula II acts as a SARM in humans with an agonist effect on some tissues while sparing the prostate or potentially antagonizing androgen related effects on the prostate. These data indicate that the compound of Formula II reduces prostate size and increases the pelvic floor muscles. Animal and human safety data indicated that the compound of Formula II has an acceptable safety profile

The present invention comprises a method of treating renal disease or treating at least one of muscle wasting, low muscle strength, or low physical function in a subject having renal disease by administering at least one compound of Formula I in a therapeutically effective amount to a subject in need thereof.

The present invention comprises methods of treating symptoms as a result of secondary hypogonadism induced by renal replacement therapy, dialysis or kidney failure in a subject having renal disease by administering at least one compound of Formula I in a therapeutically effective amount to a subject in need thereof.

The compounds of the first active ingredient as described herein are those of Formula I:

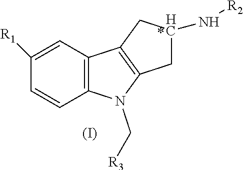

wherein the C* atom may be R, S or R/S configuration;

R₁ represents cyano, —CH═NOCH₃, —OCHF₂, or —OCF₃;

R₂ represents —COR₂ₐ or —SO₂R_{2b};

R₂ₐ represents (C₁-C₄)alkyl, (C₁-C₄)alkoxy, cyclopropyl, or —NRaRb;

R_{2b} represents (C₁-C₄)alkyl, cyclopropyl, or —NRaRb;

Ra and Rb each independently is H or (C₁-C₄)alkyl; and

R₃ represents a heteroaryl group selected from the group consisting of pyridinyl, pyrimidinyl, pyrazinyl, pyridazinyl, thiazoloy isothiazolyl, and thiadiazolyl, each optionally substituted with 1 or 2 substituents independently selected from the group consisting of methyl, ethyl, bromo, chloro, fluoro, —CHF₂, —CF₃, hydroxyl, amino and —NHCH₂CO₂H, or a pharmaceutically acceptable salt thereof.

Preferred compounds of the invention include those wherein R₂ and R₃ are any of the variables as defined herein and:

R₁ is CN, —CH═NOCH₃ or —OCF₃ or;

R₁ is CN or —CH═NOCH₃; or

R₁ is CN or

R₁ is —CH═NOCH₃.

In another embodiment, in the compounds of Formula I, R₁ and R₃ have any of the variables as defined herein and:

R₂ is —COR₂ₐ or —SO₂R_{2b} wherein R₂ₐ is (C₁-C₄)alkyl, (C₁-C₄)alkoxy, cyclopropyl, or —N(CH₃)₂ and R_{2b} is (C₁-C₄)alkyl, cyclopropyl, —N(CH₃)₂ or —N(C₂H₅)₂; or

R₂ is —COR₂ₐ or —SO₂R_{2b} wherein R₂ₐ is ethyl, isopropyl, methoxy, ethoxy, propoxy, isopropoxy, isobutoxy, tert-butoxy, cyclopropyl, or —N(CH₃)₂ and R_{2b} is methyl, ethyl, propyl, cyclopropyl, —N(CH₃)₂ or —N(C₂H₅)₂; or

R₂ is —COR₂ₐ wherein R₂ₐ is selected from ethyl, isopropyl, methoxy, ethoxy, propoxy, isopropoxy, isobutoxy, tert-butoxy, cyclopropyl, or —N(CH₃)₂; or

R₂ is —COR₂ₐ, wherein R₂ₐ is isopropyl, ethoxy, isopropoxy or cyclopropyl; or

R₂ is —COR₂ₐ wherein R₂ₐ is isopropoxy; or

R₂ is —SO₂R_{2b}, wherein R_{2b} is methyl, ethyl, propyl, cyclopropyl, —N(CH₃)₂ or —N(C₂H₅)₂; or

R₂ is —SO₂R_{2b} wherein R_{2b} is cyclopropyl or —N(CH₃)₂; or

R₂ is —SO₂R_{2b} wherein R_{2b} is —N(CH₃)₂.

In another embodiment, the compounds of Formula I include those wherein R₁ and R₃ have any of the values as recited herein and R₂ is —COR₂ₐ and the “C*” carbon center is in the S configuration; or R₂ is —SO₂R_{2b} and the “C*” carbon center is in the R configuration.

In another embodiment, the compounds used for treatment include those compounds of Formula I wherein R₁ and R₂ have any of the values recited herein and R₃ is a heteroaryl group selected from the group consisting of pyridinyl, pyrimidinyl, pyrazinyl, pyridazinyl, thiazolyl, isothiazolyl, and thiadiazolyl, each optionally substituted with one or more substituents independently selected from the group consisting of methyl, bromo, chloro, fluoro, —CHF₂, hydroxyl, amino and —NHCH₂CH₂CO₂H; or

R₃ represents 6-fluoro-pyridin-2-yl, pyridine-2-yl, 3-hydroxy-pyridin-2-yl, 6-difluoromethyl-pyridin-2-yl, 2-amino-pyridin-3-yl, 2-carboxymethylamino-pyridin-3-yl, pyrimidin-4-yl, pyrimindin-2-yl, 2-chloro-pyrimidin-4-yl, thiazol-4-yl, 2-methyl-thiazol-4-yl, 2-chloro-thiazol-4-yl, thiazol-2-yl, thiazol-5-yl, thiazol-5-yl, 4-amino-thiazol-5-yl, pyrazine-2-yl, 5-methyl-pyrazin-2-yl, 3-chloro-pyrazin-2-yl, pyridazin-3-yl, 5-bromo-isothiazol-3-yl, isothiazol yl, 4,5-dichloro-isothiazol-3-yl, or [1,2,5]thiadiazol-3-yl; or

R₃ is selected from 6-fluoro-pyridin-2-yl, pyridine-2-yl, 3-hydroxy-pyridin-2-yl, 6-difluoromethyl-pyridin-2-yl, 2-amino-pyridin-2-yl, 2-carboxymethylamini-pyridin-3-yl, thiazol-4-yl, 2-methyl-thiazol-4-yl, 2-chloro-thiazol-4-yl, thiazol-2-yl, thiazol-5-yl, 4-amino-thiazol-5-yl, pyrazine-2-yl, 5-methyl-pyrazin-2-yl, 3-chloropyrazin-2-yl, 6-methyl-pyrazin-2-yl, 3-amino-pyrazin-2-yl or 3-methyl-pyrazin-2-yl; or

R₃ is selected from 6-fluoro-pyridin-2-yl, pyridine-2-yl, 2-amino-pyridin-3-yl, thiazol-5-yl or 4-amino-thiazol-5-yl; or

R₃ is selected from pyridine-2-yl, 2-amino-pyridin-3-yl, thiazol-5-yl or 4-amino-thiazol-5-yl.

In another embodiment, the compounds for treatment in a subject having renal disease includes compounds of formula I wherein when R₂ is —COR₂ₐ, the “C*” carbon is in the S configuration; and when R₂ is —SO₂R_{2b}, the “C*” carbon is in the R configuration; R₁ is selected from cyano or —CH═NOCH₃; R₂ is selected from —COR₂ₐ or —SO₂R_{2b} wherein Rea represents (C₁-C₄)alkyl-, (C¹-C₄)alkoxy-, cyclopropyl, or —N(CH₃)₂ and R_{2b} represents (C₁-C₄)alkyl, cyclopropyl, —N(CH₃)₂ or —N(C₂H₅)₂; and R₃ represents a heteroaryl group selected from the group consisting of pyridinyl, pyrimidinyl, pyrazinyl, pyridazinyl, thiazolyl, isothiazolyl, and thiadiazolyl, each of which is independently selected from the group consisting of methyl, bromo, chloro, fluoro, —CHF₂, hydroxyl, amino, and —NHCH₂CO₂H.

In one embodiment, the compound used for treatment in a subject having renal disease is represented by Formula (I)a:

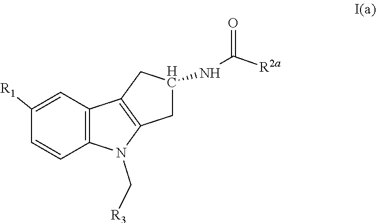

wherein,

R₁ is cyano, —CH═NOCH₃, or —OCF₃;

R^{2a} is —(C₁-C₄)alkyl, (C₁-C₄)alkoxy-, cyclopropyl or —N(CH₃)₂; and

R₃ represents a heteroaryl group selected from the group consisting of pyridinyl, pyrimidinyl, pyrazinyl, pyridazinyl, thiazolyl, isothiazolyl, and thiadiazolyl, optionally substituted with at least one of methyl, bromo, chloro, fluoro, —CHF₂, hydroxyl, amino, or —NHCH₂CO₂H.

Additional embodiments for treatment include compounds of formula I(a) wherein R₁ is cyano or —CHNOCH₃; R₂ₐ is selected from the group consisting of ethyl, isopropyl, methoxy, ethoxy, propoxy, isopropoxy, isobutoxy, tert-butoxy, cyclopropyl, or —N(CH₃)₂; and R₃ is selected from the group consisting of pyridin-2-yl, 2-amino-pyridin-3-yl, thiazol-5-yl, or 4-amino-thiazol-5-yl.

In another embodiment, the compound used in the method of the invention is a compound of formula II and pharmaceutically acceptable salts thereof:

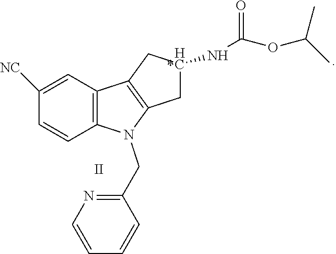

In some embodiments, the subject is undergoing dialysis. Dialysis can be hemodialysis or peritoneal dialysis. In some embodiments, the patient has stage 3 or 4 CKD.

In one embodiment, the subject selected for administration of compounds of Formula I or Formula II, is at least 40 years old. In another embodiment, the subject selected for administration of compounds of Formula I or Formula II is at least 50 years old. In another embodiment, the subject selected for administration of compounds of Formula I or Formula II is between 50 and 80 years old.

The compounds of the invention are made by alkylating a tetrahydrocyclopenta[b]indole compound with the appropriate alkylating agent of the formula R₃—CH₂—X wherein X is a leaving group (halogen) and R₃ is defined as recited herein. U.S. Pat. No. 7,968,587 which describes the synthesis of such compounds and is hereby incorporated by reference.

Compounds of the present invention may be formulated as part of a pharmaceutical composition. As such, a pharmaceutical composition comprising a compound of Formula (I), or a pharmaceutically acceptable salt thereof, in combination with a pharmaceutically acceptable carrier, diluent or excipient is an important embodiment of the invention. Examples of pharmaceutical compositions and methods for their preparation are well known in the art. See, e.g. REMINGTON: THE SCIENCE AND PRACTICE OF PHARMACY (A. Gennaro, et al., eds., 19.sup.th ed., Mack Publishing (1995)). Illustrative compositions comprising compounds of Formula (I) include, but are not limited to, a compound of Formula (I) in suspension with 1% sodium carboxymethyl cellulose, 0.25% polysorbate 80, and 0.05% Antifoam 1510.™. (Dow Corning); or a compound of Formula (I) in suspension with 0.5% methylcellulose, 0.5% sodium lauryl sulfate, and 0.1% Antifoam 1510 in 0.01N HCl (final pH about 2.5-3).

In one embodiment, the methods of treatment described herein further comprise administering a second composition (“second compound”). In another embodiment, the second compound administered comprise numerous vitamins and minerals that will improve the nutritional state of an individual having compromised renal function. The second compound of the present invention comprise carotenoids, vitamin C, vitamin D, vitamin E, vitamin K, vitamin B1, vitamin B2, vitamin B3, vitamin B5, vitamin B6, vitamin B7, vitamin B9, and vitamin B12. the second compound of the present invention comprise selenium and zinc. The second compound of the present invention include antioxidant amino acids such as L-cysteine and glutathione.

The term “carotenoids” means the tetraterpenoid family of natural substances and includes both xanthophylls and carotenes. Xanthophylls are exemplified by lutein and zeaxanthin. The carotenes include alpha-carotene, beta-carotene and lycopene.

In another embodiment, the second compound administered is extended-release calcifediol (ERC). In one embodiment, ERC is an orally administered prohormone of active vitamin D (1,25-dihydroxyvitamin D [1,25D]). In another embodiment, the ERC is synthetically manufactured as calcifediol (25-hydroxyvitamin D₃) monohydrate. In another embodiment, the ERC is (3β,5Z,7E)-9,10-secocholesta-5,7, 10(19)-triene-3,25-diol monohydrate.

In another embodiment, the second compound administered is extended-release calcifediol (ERC) having the following structure:

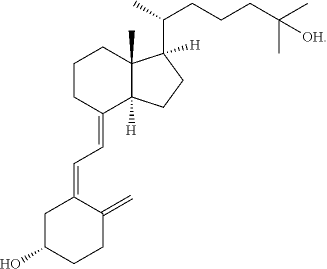 [SMILES: C=C1CC[C@H](O)C/C1=C/C=C1\CCC[C@]2(C)[C@@H]([C@H](C)CCCC(C)(C)O)CC[C@@H]12]

In another embodiment, the second compound administered includes 25-hydroxyvitamin D₂, 25-hydroxyvitamin D₃, or a combination thereof.

In another embodiment, the structures and formulations of 25-hydroxyvitamin D are described in U.S. Pat. No. 10,300,078, which is hereby incorporated by reference in its entirety. In another embodiment, the formulation of ERC is described in Cozzolino, Mario, and Markus Ketteler. “Evaluating extended-release calcifediol as a treatment option for chronic kidney disease-mineral and bone disorder (CKD-MBD).” Expert opinion on pharmacotherapy (2019): 1-13.

In one embodiment, the second administered compound is a sexual function drug. In another embodiment, the second administered compound is a phosphodiesterase type-5 (PDE-5) inhibitor.

Phosphodiesterase type-5 (PDE-5) inhibitors include, but are not limited to, sildenafil, vardenafil, or tadalafil. The latter active ingredient has been approved for erectile dysfunction. Certain drugs have been co-administered in separate dosage forms in clinical studies for the treatment of erectile dysfunction, including the co-administration of tadalafil and the compound of Formula II at particular strengths.

In one embodiment, the second administered compound is a drug typically provided to ESRD patients. These patients may have conditions that are inclusive of metabolic disorders such as diabetes or conditions inclusive of cardiovascular diseases, infectious diseases, sarcopenia, sexual dysfunction disorders or any other condition or disease related to chronic kidney diseases or disorders and/or specifically related to dialysis treatment.

In one embodiment, the second compound is co-administered with the first tetrahydrocyclopenta[b]indole compounds disclosed throughout. In another embodiment, both compounds are administered simultaneously. In another embodiment, the second compound is adminstered in a therapeutically effective amount to the subjects disclosed throughout the present application.

The invention also provides pharmaceutical compositions comprising one or more compounds of Formula I in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. It is also envisioned that the compounds of the present invention may be incorporated into transdermal patches designed to deliver the appropriate amount of the drug in a continuous fashion. The preferred dosage form is an oral capsule or tablet. A compound of Formula I or II, or a composition comprising a compound of Formula I or II can be administered by any route which makes the compound bioavailable, including oral and parenteral routes.

Recent clinical studies have surprisingly shown that there is a distinction between the preferred SARM, OPK88004, administered at a low dose (e.g. 5 mgs) versus a dose at higher strengths of 10-15 mgs/day.

In one embodiment, the dosage range for compounds of Formula I or Formula II is about 0.5 mg to about 50 mg. In one embodiment, the dosage is about 1 mg to about 5 mg. Alternatively, the dose may be in terms of mg/kg. In this format, a typical dose is about 0.02 mg/kg to about 0.1 mg/kg. For example, most patients are adult men who are 50 to 120 kg so a narrow mg/kg range might be from 0.02 mg/kg (1 mg to 50 kg patient) to 0.1 mg/kg (10 mg to 100 kg patient).

In one embodiment, the dosage range for the second compound administered is about 0.5 mg to about 50 mg. In another embodiment, the dosage range for the second compound administered is 1 mg to about 5 mg.

In one embodiment, the compounds of Formula I or Formula II are administered to a subject at a dosage of 5 mg, 15 mg, or 25 mg once daily. In one embodiment, the dosage range for the second compound administered is a dosage of 5 mg, 15 mg, or 25 mg once daily.

In one embodiment, the compounds of Formula I or Formula II are administered to a subject at a dosage of 1-5 mg, once daily. In one embodiment, the dosage range for the second compound administered is a dosage of 1-500 μg (micrograms) of 25(OH)D3 in a sustained release dosage form once daily. The controlled release calcifdiol may also be administered at doses of 300 to 1000 μgs per week to dialysis patients on a dosing schedule of once per week or three times per week, four times per week or two times per week or on a daily basis.

In another embodiment the compounds of Formula I or II is administered to a subject in a dose ranging from 0.0001 to 5 mg per day. In another embodiment the compounds of Formula I or II is administered to a subject in a dose ranging from 5 to 15 mg per day. In another embodiment the compounds of Formula I or II is administered to a subject in a dose ranging from 15 to 25 mg per day.

In another embodiment the compounds of Formula I or II is administered to a subject in a dose ranging from 0.0001 to 5 mg per day. In another embodiment the compounds of Formula I or II is administered to a subject in a dose ranging from 1 to 5 mg per day.

In one embodiment, the dosage range for the second compound administered is at a dose ranging from 0.0001 to 5 mg per day. In another embodiment, the dosage range for the second compound administered is at a dose ranging from (i) 5 mg to 15 mg or (ii) 15 mg to 25 mg per day.

In one embodiment, the dosage range for the second compound administered is at a dose ranging from 1 to 500 μg per day. In another embodiment, the dosage range for the second compound administered is at a dose ranging from 1 to 1000 μg per day or 1 to 1000 μg/week.

In another embodiment the compounds of Formula II is administered to a subject in a dose ranging from 0.0001 to 5 mg per day. In another embodiment the compounds of Formula II is administered to a subject in a dose ranging from 5 to 15 mg per day. In another embodiment the compounds of Formula II is administered to a subject in a dose ranging from 15 to 25 mg per day.

In another embodiment the compounds of Formula II is administered to a subject in a dose ranging from 0.0001 to 5 mg per day. In another embodiment the compounds of Formula II is administered to a subject in a dose ranging from 1 to 5 mg per day.

In one embodiment, the compounds of Formula I or Formula II are administered once daily for a period of at least four weeks. In another embodiment, the compounds of Formula I or Formula II are administered once daily for a period of at least eight weeks. In another embodiment, the compounds of Formula I or Formula II are administered once daily for a period of at least twelve weeks. In another embodiment, the compounds of Formula I or Formula II are administered once daily for a period of at least sixteen weeks. In another embodiment, the compounds of Formula I or Formula II are administered once daily for a period of at least twenty weeks. In another embodiment, the compounds of Formula I or Formula II are administered once daily for a period of up to six months. In another embodiment, the compounds of Formula I or Formula II are administered once daily for a period of up to two years or more.

For preparing solid compositions such as tablets, the principal active ingredient (the compound of Formula I or II) is mixed with a pharmaceutically acceptable carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture for a compound of the present invention, or a pharmaceutically acceptable salt thereof. When referring to these preformulation compositions as homogeneous, it is meant that the active ingredient is dispersed evenly throughout the composition so that the composition may be easily subdivided into equally effective unit dosage forms such as tablets, pills and capsules. This solid pre-formulation composition is then subdivided into unit dosage forms of the type described above containing from 0.1 to about 500 mg of the active ingredient. Typical unit dosage forms contain from 1 to 10 mg, for example, 1, 2, or 5 mg, of the active ingredient. The tablets or pills of the composition can be coated or otherwise compounded to provide a dosage affording the advantage of prolonged action. For example, the tablet or pill can comprise an inner dosage and an outer dosage component, the latter being in the form of an envelope over the former. The two components can be separated by an enteric layer which, serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

In one embodiment, the compounds of Formula I or Formula II are administered orally in a gelatin capsule. In another embodiment, each gelatin capsule for oral administration contains the compounds of Formula I or Formula II, inactive ingredients, pregelatinized starch and dimethicone. In one embodiment, the gelatin capsules containing at least the compounds of Formula I or Formula II have at least one of the data points described in Table 2 below as well as a range of 30% on each side of each data point selected from the group consisting of the following properties: assay, un-specified impurity, total impurities, water activity, dissolution.

TABLE 2

| Test | Acceptance Criteria | Result |
|---|---|---|
| Physical | Blue Capsules | Pass |
| Appearance | | |
| (Visual) | | |
| Package | Report Results | Pass |
| Characteristics | | |
| (Visual) | | |
| Assay | NLT 90.0% and NMT | 100.0% to 101.6% |
| (HPLC) | 110.0% of the label | |
| | claim | |
| Any | Impurities: | 0.49% to 0.58% |
| Unspecified | Individual | |
| Impurity | Percent | |
| (HPLC) | | |
| Total Impurities | Impurities: Total | 0.49% to 0.58% |
| (HPLC) | Percent | |
| Water Activity | Report results | 0.29 aw to 0.32 aw |
| Dissolution | Meets USP | Pass |
| (Apparatus | <711> with | Low = 99 to 101 % |
| II/HPLC) | Q = 75% at | High = 102 to 104% |
| | 45 minutes | Avg. = 101 to 102% |
| | (No. of Units = 6) | |

In one embodiment, the gelatin capsules containing at least the compounds of Formula I or Formula II has a potency of at least 90.0% when measured using an assay. In another embodiment, the gelatin capsules containing at least the compounds of Formula I or Formula II has a potency of not more than 110.0% when measured using an assay. In another embodiment, the gelatin capsules containing at least the compounds of Formula I or Formula II meets the requirements set forth in <905> of the United States Pharmacopeia Convention. In another embodiment, the gelatin capsules containing at least the compounds of Formula I or Formula II has the following microbial limits: TMAC<1000 cfu/g, TYMC<100 cfu/g; Absence of Escherichia coli/lg.

The compounds of the present invention are useful when formulated in the form of a pharmaceutical injectable dosage, including a compound described and claimed herein in combination with an injectable carrier system. As used herein, injectable and infusion dosage forms (i.e., parenteral dosage forms) include, but are not limited to, liposomal injectables or a lipid bilayer vesicle having phospholipids that encapsulate an active drug substance. Injection includes a sterile preparation intended for parenteral use.

Five distinct classes of injections exist as defined by the USP: emulsions, lipids, powders, solutions and suspensions. Emulsion injection includes an emulsion comprising a sterile, pyrogen-free preparation intended to be administered parenterally. Lipid complex and powder for solution injection are sterile preparations intended for reconstitution to form a solution for parenteral use. Powder for suspension injection is a sterile preparation intended for reconstitution to form a suspension for parenteral use. Powder lyophilized for liposomal suspension injection is a sterile freeze dried preparation intended for reconstitution for parenteral use that is formulated in a manner allowing incorporation of liposomes, such as a lipid bilayer vesicle having phospholipids used to encapsulate an active drug substance within a lipid bilayer or in an aqueous space, whereby the formulation may be formed upon reconstitution. Powder lyophilized for solution injection is a dosage form intended for the solution prepared by lyophilization (“freeze drying”), whereby the process involves removing water from products in a frozen state at extremely low pressures, and whereby subsequent addition of liquid creates a solution that conforms in all respects to the requirements for injections. Powder lyophilized for suspension injection is a liquid preparation intended for parenteral use that contains solids suspended in a suitable fluid medium, and it conforms in all respects to the requirements for Sterile Suspensions, whereby the medicinal agents intended for the suspension are prepared by lyophilization. Solution injection involves a liquid preparation containing one or more drug substances dissolved in a suitable solvent or mixture of mutually miscible solvents that is suitable for injection. Solution concentrate injection involves a sterile preparation for parenteral use that, upon addition of suitable solvents, yields a solution conforming in all respects to the requirements for injections. Suspension injection involves a liquid preparation (suitable for injection) containing solid particles dispersed throughout a liquid phase, whereby the particles are insoluble, and whereby an oil phase is dispersed throughout an aqueous phase or vice-versa. Suspension liposomal injection is a liquid preparation (suitable for injection) having an oil phase dispersed throughout an aqueous phase in such a manner that liposomes (a lipid bilayer vesicle usually containing phospholipids used to encapsulate an active drug substance either within a lipid bilayer or in an aqueous space) are formed. Suspension sonicated injection is a liquid preparation (suitable for injection) containing solid particles dispersed throughout a liquid phase, whereby the particles are insoluble. In addition, the product may be sonicated as a gas is bubbled through the suspension resulting in the formation of microspheres by the solid particles.

The parenteral carrier system includes one or more pharmaceutically suitable excipients, such as solvents and co-solvents, solubilizing agents, wetting agents, suspending agents, thickening agents, emulsifying agents, chelating agents, buffers, pH adjusters, antioxidants, reducing agents, antimicrobial preservatives, bulking agents, protectants, tonicity adjusters, and special additives.

As appreciated by one of skill in the art, physiological disorders may present as a “chronic” condition, or an “acute” episode. The term “chronic”, as used herein, means a condition of slow progress and long continuance. As such, a chronic condition is treated when it is diagnosed and treatment continued throughout the course of the disease. Conversely, the term “acute” means an exacerbated event or attack, of short course, followed by a period of remission. Thus, the treatment of disorders contemplates both acute events and chronic conditions. In an acute event, compound is administered at the onset of symptoms and discontinued when the symptoms disappear. As described above, a chronic condition is treated throughout the course of the disease.

One of skill in the art will appreciate that particle size can affect the in vivo dissolution of a pharmaceutical agent which, in turn, can affect absorption of the agent. “Particle size” as used herein, refers to the diameter of a particle of a pharmaceutical agent as determined by conventional techniques such as laser light scattering, laser diffraction, Mie scattering, sedimentation field flow fractionation, photon correlation spectroscopy, and the like. Where pharmaceutical agents have poor solubility, small or reduced particle sizes may help dissolution and, thus, increase absorption of the agent. Amidon et al., Pharm. Research, 12; 413-420 (1995). As described in U.S. Pat. No. 7,968,587 for the SARMs of Formula I, particles can be reduced in size by methods that include milling, grinding, micronization or by other methods known in the art. Another method for controlling particle size involves preparing the pharmaceutical agent in a nanosuspension. A particular embodiment of the present invention comprises a compound of Formula (I), or a pharmaceutical composition comprising a compound of Formula (I), wherein said compound has an average particle size less than about 20 μm or a d₉₀ particle size (i.e. the maximal size of 90% of the particles) of less than about 50 μm. A more particular embodiment comprises a compound of Formula I having an average particle size less than about 10 μm or a d₉₀ particle size of less than about 30 μm. The active ingredients may have a particle size that affects the dissolution profile of a pharmaceutical agent. Particle size, as used herein, means the diameter of a particle of active pharmaceutical ingredient as determined by light scattering or other conventional techniques.

As used herein the term “effective amount” refers to the amount or dose of a compound of Formula (I) which, upon single or multiple dose administration to the patient, provides the desired effect in the patient under diagnosis or treatment. An effective amount can be readily determined by the attending diagnostician, as one skilled in the art, by considering a number of factors such as the species of mammal; its size, age, and general health; the specific disease involved; the degree or severity of the disease; the response of the individual patient; the particular compound administered; the mode of administration; the bioavailability characteristics of the preparation administered; the dose regimen selected; and the use of any concomitant medications. An effective amount of a prohormone such as 25-hydroxyvitamin D₃ refers to an amount of, for example, a controlled release dosage form having about 10 to 1,000 μg of such active sufficient to treat vitamin D insufficiency and to lower PTH and treat secondary hyperparathyroidism in such ESRD patients (e.g. raise serum 25(OH)D3 levels to above 30 to 60 ng/mL while also lowering iPTH by about 30 percent) while also not inducing or causing hypercalcemia or other toxicity.

### EXAMPLES

### Example 1: In Vitro Pharmacology of the Compound of Formula II

In vitro studies demonstrated that the compound of Formula II is a potent and selective modulator of the hAR with potent agonist activity in cell-based assays and no significant cross reactivity against other nuclear hormone receptors or known drug targets across various platforms. The compound of Formula II is a selective ligand for the hAR with an inhibition constant (Ki) of 1.95 nM, and a cell-based median effective concentration (EC50) of 1.25 nM, with demonstrated agonist activity. The binding affinity for hAR compared to other nuclear hormone receptors was >500-fold (see Table 3).

TABLE 3

| Assay | TT701 |
|---|---|
| AR Ki (nM) | 1.95 |
| AR C2C12 | 1.25 |
| EC50 (nM) | |
| ER, GR, PR | >3,000 |
| MR(IC50/Ki)(nM) | |
| Muscle LA ED | 3 mpk |
| mg/kg | |
| Bone Eff (BM) | 2 mpk |
| Rat uterine risk | 20 mpk |
| Rat SV/Prost risk mgs/kg | >30 mpk |
| Rat F % | 63 |
| Dog F % | 58 |
| MOS (AUC) | 28X_{dog} + 71X_{cat} |

### Example 2: Structural, Chemical, and Pharmacological Characteristics of the Compound of Formula II

Structural Characteristics of the Compound of Formula II:

The compound of Formula II belongs to a nonsteroidal THCI scaffold that is structurally distinct from the cholesterol-derived steroidal scaffolds. The compound of Formula II has weak affinity to serum hormone binding globulin (none detected at 10 μM) and is not metabolized by 17-beta-Hydroxysteroid Dehydrogenase Type 2 class of enzymes. The x-ray crystallography structure of the compound of Formula II-bound AR illustrates some key differences in the contact sites within the active pocket relative to that of dihydrotestosterone-bound AR.

### Example 3: In Vitro Activity of the Compound of Formula II on LnCAP Cells

The compound of Formula II has weak agonist activity in in vitro prostate LnCAP cells (androgen-sensitive human prostate adenocarcinoma cells) being at least 46 fold weaker than the synthetic testosterone R188. Comparisons of the compound of Formula II with the synthetic Testosterone R1881, showed that in vitro using human prostate cancer cells the LY compound is less androgenic than R1881. In contrast the biochemical binding affinity to the human Androgen receptor (Ki in nM) is only modestly reduced. The ability of the compound of Formula II to bind to the Androgen receptor and yet have a very weak agonist activity in gene expression compared to the synthetic Testosterone R188, suggests that the presence of the compound of Formula II may interfere or reduce AR activity of endogenous Testosterone (see Table 4).

TABLE 4

| | AR Ki | LnCAP Gene Expression EC50 (nM) | | |
|---|---|---|---|---|
| | (nM) | PSA | AR | CLUSTERIN |
| R1881 | 0.38 | 0.034 | 0.035 | 0.37 |
| Compound | 1.95 | 2.64 | 1.64 | >100 |
| of Formula II | | | | |
| Fold Diff | ~5× | ~77× | ~46× | ~270× |

### Example 4: Anabolic and Androgenic Effects of the Compound of Formula II in Different Animal Studies

The effect of short and long term treatment of the compound of Formula II on prostate size of rats and dogs was studied. As part of the toxicology program, rats and dogs were treated with escalating doses of the compound of Formula II for 1, 3, 6 or 12 months depending on the study or species and examined for prostate size and histologically for prostate atrophy. The data is shown in Tables 5 and 6 below.

TABLE 5
Rat Data

| Treatment duration | | | | | | | | | |
|---|---|---|---|---|---|---|---|---|---|
| (months) | 1+ | | | 6+ | | | (3 months) | | |
| Dose (mg/kg/day) | 15 | 150 | 1500 | 15 | 150 | 1500 | 3 | 30 | 1000 |
| Prostate weight | “” | “” | “” | “” | ^{↓}30% | ^{↓}34% | “” | ^{↓}23% | ^{↓}39% |
| (mean % decrease) | | | | | | | | | |
| Prostate atrophy | “” | “” | “” | “” | | 4/15 | “” | “” | “” |
| (no affect/no examined) | | | | | | | | | |
| Male group mean AUC₀₋₂₄ | 35733 | 72283 | 102337 | 15902 | 34132 | 82690 | 10954 | 49734 | 56009 |

TABLE 6
Dog Data

| Treatment duration | | | | | | | | | |
|---|---|---|---|---|---|---|---|---|---|
| (months) | 1+ | | | 6+ | | | 12 | | |
| Dose (mg/kg/day) | 3 | 30 | 150 | 3 | 30 | 150 | 3 | 10 | 100 |
| Prostate weight | “” | “” | “” | ^{↓}63% | ^{↓}66% | ^{↓}75% | ^{↓}60 | ^{↓}62% | ^{↓}80% |
| (mean % decrease) | | | | | | | | | |
| Prostate atrophy | “” | “” | 1/4 | 4/4 | 4/4 | 4/4 | 2/4 | 3/4 | 4/4 |
| (no affect/no examined) | | | | | | | | | |
| Male group mean AUC₀₋₂₄ | 17984 | 61674 | 46528 | 6492 | 44448 | 53032 | 3641 | 13408 | 22582 |
| mean AUC₀₋₂₄ | | | | | | | | | |

“′” no effect observed. Vehicle 80% PEG 1350, 20% vitamin E TPGS (w/v)

These data demonstrate that treatment with the compound of Formula II at different doses decreased prostate weight in both rats and dogs. The loss in weight was more pronounced in dogs relative to rats, and in dogs decreases of 60-75% were observed by 3 months. In addition, histological examination showed prostate atrophy in 50 to 100% of the animals treated for 6-12 months.

Importantly, there is a lack of androgenic effect on the prostate with very high doses of the compound of Formula II in both rats and dogs. The reductions in the weight and/or atrophy of the prostate are consistent with the antagonistic properties of the compound of Formula II on androgenic effects on the prostate.

### Example 5: Anabolic and Androgenic Effects of the Compound of Formula II in the Rat Osteopenic Orchidectomized (ORX) Model

An orchidectomized rat model was used to examine the anabolic and androgenic effects of the compound of Formula II in the absence of endogenous testosterone in animals. Orchidectomized (ORX) and sham-operated Wistar male rats were used (orchidectomized at 8 weeks of age and allowed to waste for 4 weeks). The rats were maintained on a 12 hr light/dark cycle at 22° C. with ad lib access to food (TD 5001 with 0.95% Ca and 0.67% P, Teklad, Madison, Wis.) and water. Rats were randomized and placed into treatment groups (n=6) based on body weight. Route of administration for all groups except testosterone enanthate (TE was given sub-cutaneously) was oral. At the end of 2 weeks of daily rats were euthanized, weighed & tissue harvested. Levator ani, prostates, and seminal vesicles were collected from each animal. Results are plotted as means±SE.

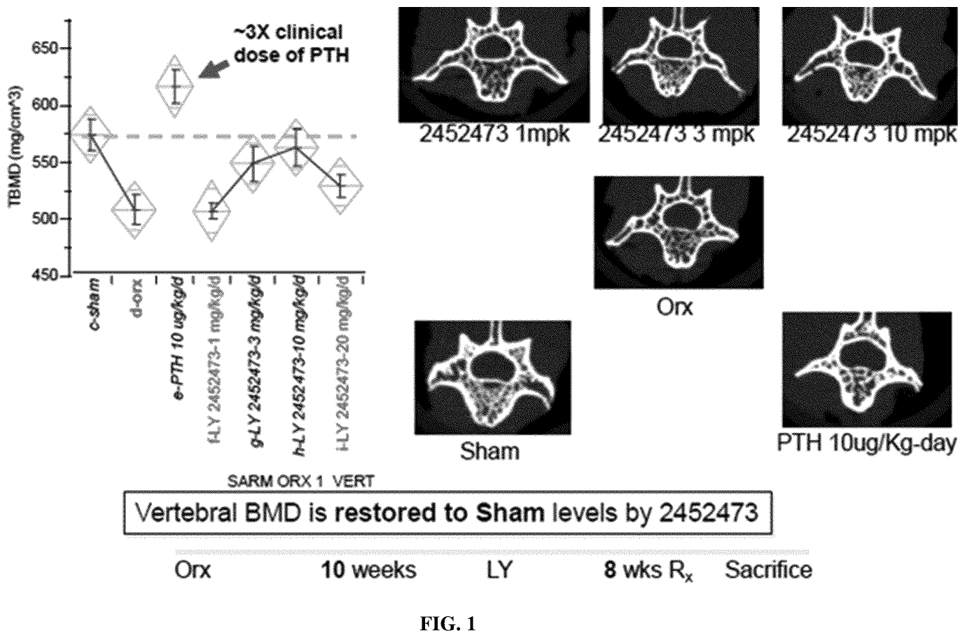

The results are illustrated in FIG. 1, daily oral administration of the compound of Formula II led to a dose-dependent increase in vertebral bone mineral content (BMC), cross-sectional area, and bone mineral density (BMD). A significant increase in the bone anabolic biomarkers, rat procollagen type 1 amino-terminal propeptide (P1NP), and periosteal alkaline phosphatase, were observed after 2 and 8 weeks of treatment.

### Example 6: Anabolic Effects of the Compound of Formula II on the Levator Ani- and Bulbocavernous Muscle in the ORX Model

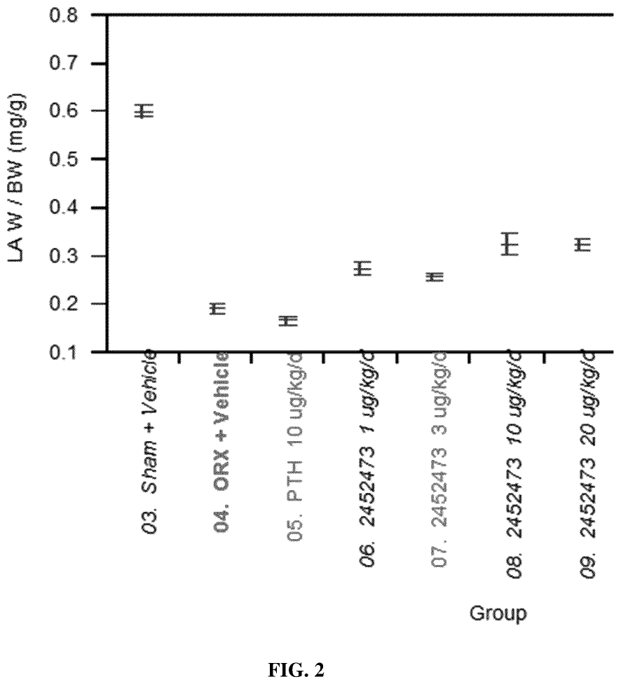

The anabolic effects of the compound of Formula II on the Levator ani- and bulbocavernous muscle in the delayed ORX model were examined. Following the androgen deficiency in the ORX model, the Levator ani- and bulbocavernous muscle weights decreased in size. Following treatment with the compound of Formula II for 8 weeks, the Levator ani- and bulbocavernous muscle weights increased significantly with doses of 1 to 3 mg/kg/day doses. These data indicate that the compound of Formula II has positive anabolic effects on the Levator ani and bulbocavernous muscle in testosterone deficient animals. The results are graphically represented in FIG. 2 and tabulated in Table 7.

TABLE 7

| Subject | Dose (ug/kg/d) | Number | Mean | Std Error Mean |
|---|---|---|---|---|
| 3 | Sham + Vehicle | 7 | 0.599700 | 0.01204 |
| 4 | ORX + Vehicle | 7 | 0.192057 | 0.01180 |
| 5 | PTH 10 | 6 | 0.166467 | 0.00913 |
| 6 | compound of | 6 | 0.274917 | 0.01522 |
| | Formula II-1 | | | |
| 7 | compound of | 7 | 0.257529 | 0.00773 |
| | Formula II-3 | | | |
| 8 | compound of | 7 | 0.323743 | 0.02138 |
| | Formula II-10 | | | |
| 9 | compound of | 7 | 0.325043 | 0.01218 |
| | Formula II-20 | | | |

The compound of Formula II demonstrated a robust indicator of muscle and bone loss associated with androgen deprivation. In 2 and 8 week studies, the compound of Formula II demonstrated the ability to increase intrapelvic skeletal muscle wet weight, restore bone loss, and improve bone strength in the cortical site and femoral neck.

### Example 7: The Androgenic Effects of the Compound of Formula II in the ORX Rat Model

FIGS. 1 and 2 illustrate the effect of the compound of Formula II treatment for 2 or 8 weeks on the prostate weight and seminal vesicles in the delayed ORX rat model. The weights and seminal vesicals of orchidectomized rats decreased significantly. Treatment of delayed ORX rats with increasing doses of the compound of Formula II had no effect on the prostate or seminal vesical weights. These data clearly demonstrate that doses of up to 20-30 mg/kg do not have androgenic activity on the prostate.

Anabolic activity on muscle and bone induced by the compound of Formula II treatment was observed in the absence of androgenic related effects on prostate weight and histology or on seminal vesicles weight that confirmed the ‘prostate sparing’ effects of the compound of Formula II.

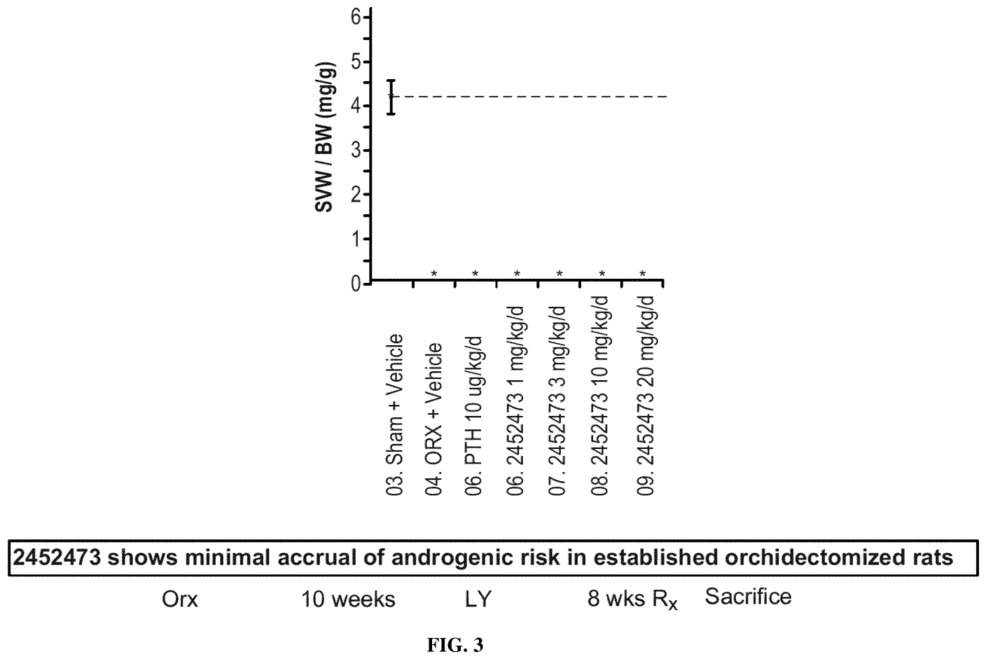

The data in FIG. 3 shows that TT701 treatment in the ORX rat shows minimal accrual SV/Prostate risk. Thus, anabolic activity on muscle and bone induced by TT701 treatment was observed in the absence of androgenic related effects on prostate weight and histology or on seminal vesicles weight, which confirms the prostate sparing effects of TT701.

### Example 8: Effect of the Compound of Formula II on Testosterone Induced Prostate Growth in Delayed ORX Rat Model

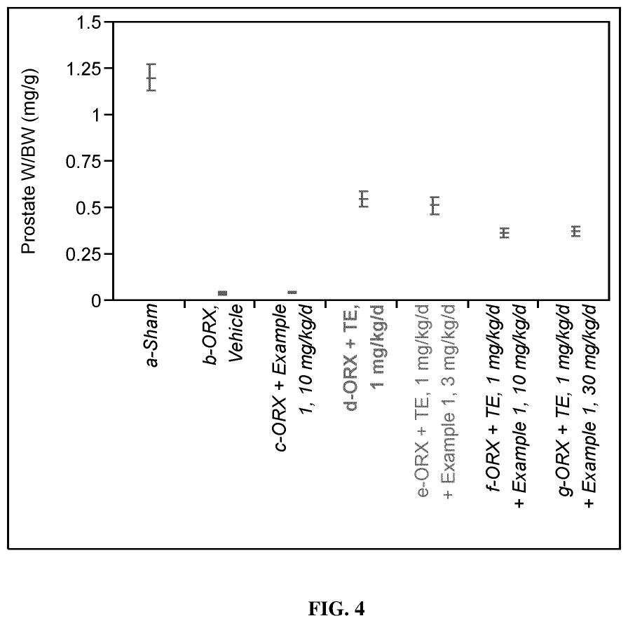

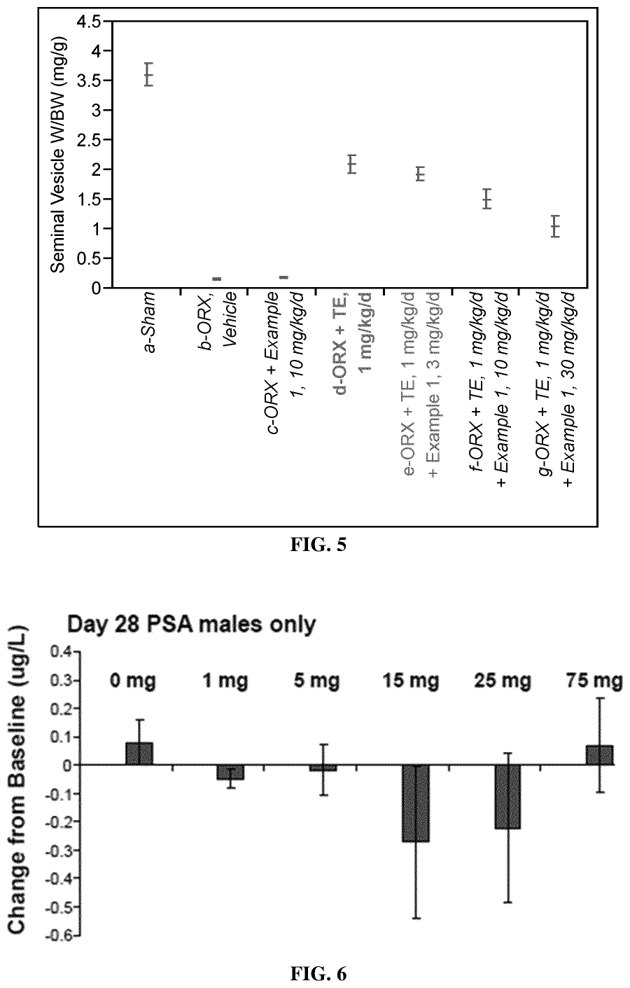

FIGS. 4 and 5 illustrate the effect of orchidectomized rats treated with Testosterone (1 mg/k/d) alone for 2 weeks showed an increase in prostate weight and seminal vesical weight, respectively. The combination with Testosterone Enanthate (TE) (1 mg/kg/d) and the compound of Formula II at 3, 10 and 30 mg/kg/d demonstrated a decreasing prostate weight and seminal vesical weights normalized to body weight, compared to the prostate and seminal vesical weights induced by TE alone after 2 weeks of co-treatment. These data demonstrated that the compound of Formula II antagonized the androgenic effects of testosterone on the prostate. Table 8 contains the data on the effect on prostate weight of the treatment with TE and the compound of Formula II. Table 9 contains the date on the effect on seminal vesical weight of the treatment with T and the compound of Formula II.

TABLE 8
Means Comparisons of prostate weights (Comparisons with a control
using Dunnett's Method Control Group = d-ORX + TE, 1 mg/kg/d

| \|d\| | | | |
|---|---|---|---|
| 2.69715 | Alpha | | |
| \|d\| | 0.05 | | |
| Group | Alpha | Abs(Dif)- | |
| No | Group | LSD | p-Value |
| 1 | Sham | 0.509 | <.0001* |
| 2 | ORX + TE, 1 mg/kg/d | −0.15 | 1.0000 |
| 3 | ORX + TE, 1 mg/kg/d + | −0.11 | 0.9774 |
| | Example 1, 3 mg/kg/d | | |
| 4 | ORX + TE, 1 mg/kg/d + | 0.025 | 0.0167* |
| | Example 1, 30 mg/kg/d | | |
| 5 | ORX + TE, 1 mg/kg/d + | 0.036 | 0.0099* |
| | Example 1, 10 mg/kg/d | | |
| 6 | ORX, Vehicle | 0.356 | <.0001* |
| 7 | ORX + Example 1, 10 mg/kg/d | 0.357 | <.0001* |

Positive values show pairs of means that are significantly different than TE alone group

TABLE 9
Means Comparisons of seminal vesicle
wet weights (Comparisons with a control using
Dunnett's Method): Control Group = d-ORX + TE, 1 mg/kg/d

| \|d\| | | | |
|---|---|---|---|
| 2.69715 | Alpha | | |
| Group | 0.05 | Abs(Dif)- | |
| No | Group | LSD | p-Value |
| 1 | Sham | 0.979 | <.0001 |
| 2 | ORX + TE, 1 mg/kg/d | −0.52 | 1.0000 |
| 3 | ORX + TE, 1 mg/kg/d + | −0.34 | 0.8628 |
| | Example 1, 3 mg/kg/d | | |
| 4 | ORX + TE, 1 mg/kg/d + | 0.078 | 0.0187 |
| | Example 1, 10 mg/kg/d | | |
| 5 | ORX + TE, 1 mg/kg/d + | 0.536 | <.0001 |
| | Example 1, 30 mg/kg/d | | |
| 6 | ORX + Example 1, 10 mg/kg/d | 1.411 | <.0001 |
| 7 | ORX, Vehicle | 1.422 | <.0001 |

Positive values show pairs of means that are significantly different.

### Example 9: Clinical Studies with the Compound of Formula II

Six clinical studies were completed with the compound of Formula II (Table 10): five Phase 1 clinical trials (Studies GPBA, GPBC, GPBG, GPBF, and GPEA) and two Phase 2 clinical trials (Studies GPEC and SAR-202). A total of 431 subjects have been exposed to the compound of Formula II in these completed clinical studies. In Studies GPEA and GPEC, the compound of Formula II was orally co-administered with tadalafil.

TABLE 10
Completed Clinical Studies with the compound of Formula II

| Study | |
|---|---|
| Number | |
| (Section) | Study Design (N) |
| GPBA | Phase 1, randomized, placebo-controlled, |
| | double-blind, single-dose (5, 25, 125, 250, |
| | 500 or 1000 mg/day), incomplete-crossover, |
| | dose-escalation study in healthy men and |
| | postmenopausal women (N = 29) |
| GPBC | Phase 1, randomized, placebo-controlled, |
| | double-blind, multiple-dose (1, 5, 15, 25, |
| | 75 mg/day), dose-escalation study of |
| | the compound of Formula II in |
| | healthy subjects for 28 days (N = 44) |
| GPBG | Single-center, open-label, 2-period, fixed- |
| | sequence study to assess the effects of |
| | multiple oral doses of the compound of |
| | Formula II (5 mg/day) on simvastatin and |
| | simvastatin acid activity in healthy subjects (N = 16) |
| GPBF | Phase 1, open-label, 4-period crossover |
| | bioavailability study conducted in healthy male |
| | subjects to evaluate the pharmacokinetics of the |
| | compound of Formula II and tadalafil (N = 24) |
| GPEA | Phase 1, randomized, open-label, 4-period |
| | crossover bioavailability study conducted in healthy |
| | male subjects to evaluate the pharmacokinetics of the |
| | compound of Formula II and tadalafil (N = 24) |
| GPEC | Phase 2, randomized, double-blind, parallel, |
| | controlled clinical trial in patients with ED who |
| | had an incomplete response to tadalafil; 5 dosing |
| | arms: the compound of Formula II 1 mg or 5 |
| | mg + tadalafil 5 mg, the compound of Formula |
| | II 5 mg, tadalafil 5 mg, tadalafil 10 mg, |
| | for 12 weeks (N = 234) |
| SAR-202 | Phase 2, randomized, double-blind, parallel, |
| | controlled clinical trial in patients with BPH of |
| | Formula II (15 and 25 mg/day) for 16 weeks (N = 114) |

### Example 10: Safety Assessment of TT701

Study GPBA detected a QT signal using a concentration-response analysis which showed a statistically significant positive relationship between TT701 and QTcF prolongation. At TT701 concentrations found with doses of 250 mg or greater, the mean QTcF prolongation was greater than 10 msec. The risk for clinically significant QT prolongation at doses <250 mg can be excluded. Review of ECG data in study GPEC did not show evidence of clinically meaningful changes associated with TT701.

In study GPEC, treatment with OPK-88004 was associated with decreased HDL cholesterol levels and apolipoprotein A1, whereas observed decreases in total cholesterol, triglycerides and LDL cholesterol levels were not considered clinically meaningful.

In clinical studies with TT701, no significant increases in total bilirubin, GGT, or alkaline phosphatase were observed at any dose. Transient elevation of liver transaminases (ALT or AST) observed in 14 subjects in phase 1 studies were not considered to be clinically significant by the investigator, and none were captured as AEs. In a phase 2 study, three subjects had transient abnormal AST or ALT, with levels in two subjects receiving OPK-88004 being >2×ULN and in one subject being >3×ULN. None of the elevations were considered to be clinically significant by the investigator and none were captured as AEs. All of the subjects with elevated liver transaminase levels completed the respective study.

In Study GPEC, endocrine-related parameters evaluation measured in the 12-week study in elderly men included total and free testosterone, SHBG, estradiol, follicle-stimulating hormone (FSH), and LH and semen analysis. A clinically meaningful decrease was observed for testosterone, accompanied by a reduction of SHBG,

TABLE 11
OPK-88004 Noncompartmental Pharmacokinetic Parameters following
a Single Oral Dose of OPK-88004 to Healthy Subjects (Study GPBA)
Geometric Mean (CV %)

| | 5 mg | | 25 mg | | 125 mg | | 250 mg | | 500 mg | | 1000 mg | |
|---|---|---|---|---|---|---|---|---|---|---|---|---|
| N | 8 | | 7 | | 7 | | 7 | | 7 | | 6 | |
| Cₘₐₓ(ng/mL) | 25.9 | (53) | 123 | (34) | 428 | (36) | 589 | (70) | 817 | (26) | 910 | (39) |
| Tₘₐₓ^{a}(h) | 5.00 | (3.00-6.00) | 4.00 | (3.00-5.00) | 5.00 | (4.00-5.00) | 5.00 | (3.00-10.02) | 4.00 | (3.00-5.00) | 4.50 | (2.00-10.02) |
| T_{1/2}^{b}(h) | 27.1 | (18.1-53) | 26.6 | (17.5-35.0) | 39.4^{c} | (14.4-114) | 31^{c} | (17.3-65.0) | 55.l^{d} | (22.7-138) | 41.7 | (21.1-79.9) |
| AUC₍₀₋₎ | 458 | (36) | 2110 | (20) | 8590^{c} | (111) | 17500^{c} | (29) | 29400^{d} | (68) | 24100 | (86) |

Abbreviations: Cₘₐₓ = maximum observed drug concentration; tₘₐₓ = time of maxium observed drug concentration; AUC₍₀₋₎ = area under the concentration-time curve from 0 to infinity; ^{a}Median (range); ^{b}Geometric mean (range); ^{c}n = 6; ^{d}n = 5.

### Example 11: The Effect of the Compound of Formula II on Prostate Antigen

In Study GPBC, the PSA test indicated a neutral to negative effect (decreasing PSA) on the prostate in response to increased dosing in the dose escalation study in healthy subjects (1 mg to 75 mg daily dose for 28 days) of the compound of Formula II. The results of this study are illustrated in FIG. 6.

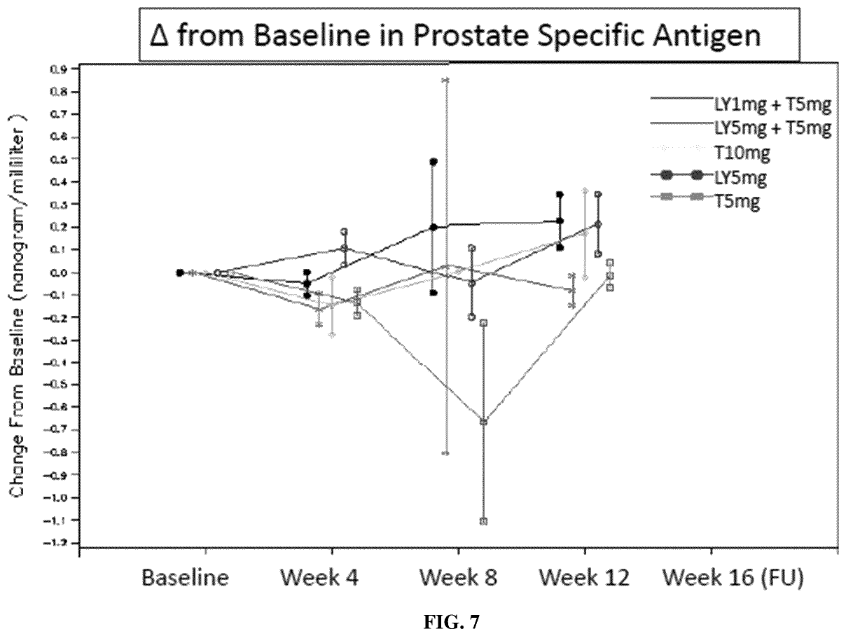

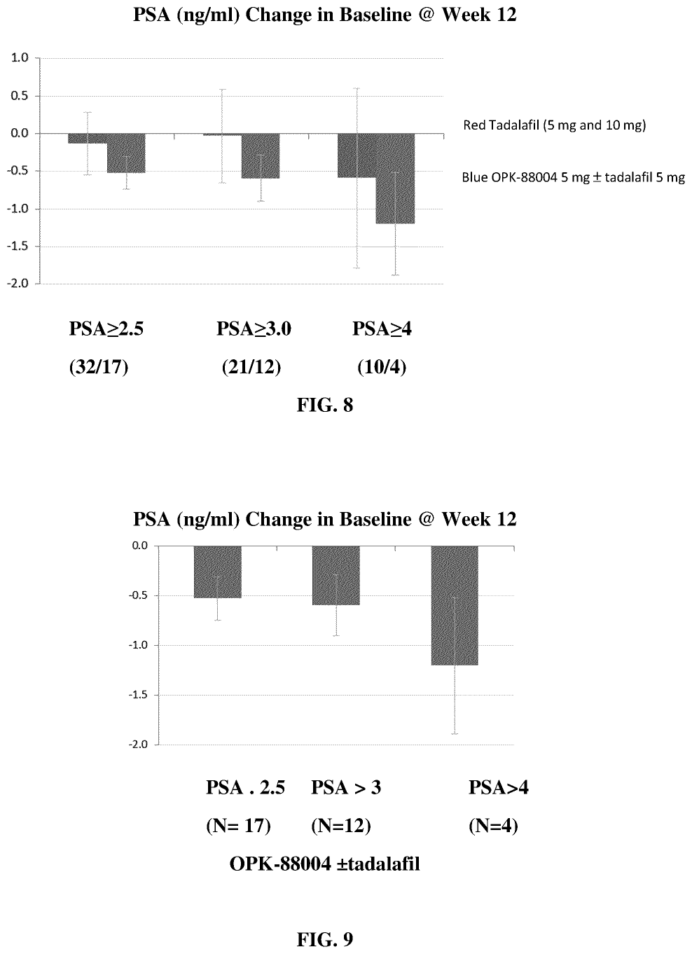

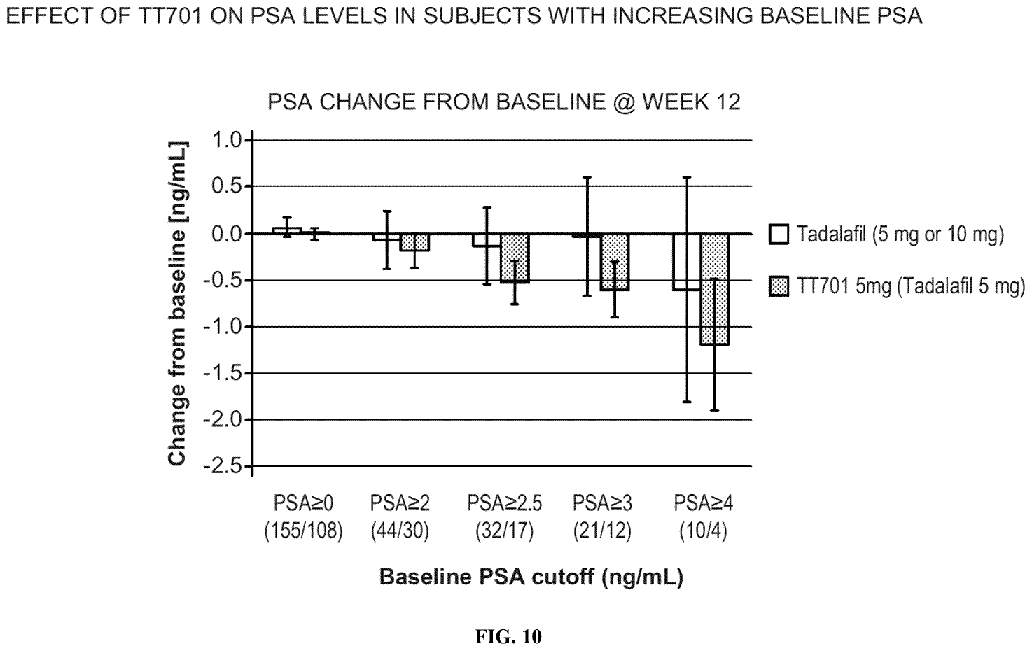

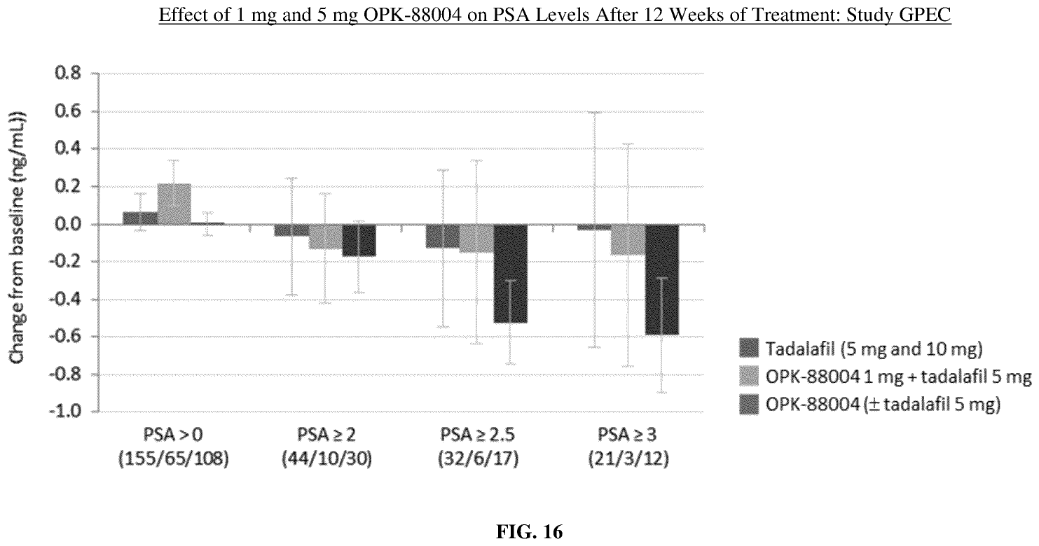

In Study GPEC, treatment with the compound of Formula II at doses of 1 mg and 5 mg for 12 weeks was not associated with increases in PSA. The results of this study are illustrated in FIG. 7. Also in this study in healthy aging males who had symptoms of erectile dysfunction and were failing on tadalafil treatment, TT701 (OPK-88004, 5 mgs) alone or in combination with 5 mgs or 10 mgs of tadalafil and in patients having PSA levels of greater than about 2.5 ng/mL, unexpectedly showed a significant decrease in PSA levels relative to baseline. (see FIG. 8 and FIG. 9). FIG. 10 shows the changes in PSA levels in patients having symptoms of erectile dysfunction and who were failing tadalafil treatment being administered OPK-88004 (TT701) alone or combination with tadalafil (5 mgs alone or 5 mg/5 mg combination) where the PSA horizontal scale is broadened. This figure shows you begin to see a decrease in PSA levels at about 2.0 PSA upon treatment with OPK-88004. FIG. 16 shows the changes in PSA levels in patients being administered tadalafil alone (5 mg and 10 mg) or OPK-88004 (TT701) (1 mg or 5 mg) in combination with tadalafil (5 mg) after 12 weeks of treatment.

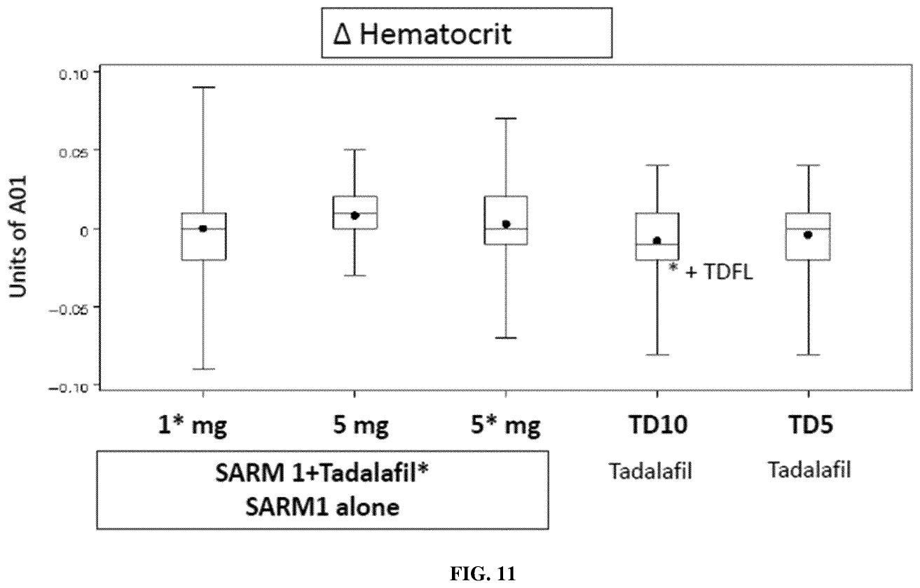

To date, no immunotoxicity safety signal has been observed. It is believed that because the compound of Formula II is a small molecule, it is not expected to be immunogenic, and an immunogenicity assay has not been developed. Androgen-induced erythrocytosis and resulting polycythemia is thought to be a significant limitation to androgen therapy, and has been shown to manifest in the first 3 months of treatment. A statistical analysis of hemoglobin and hematocrit showed no increases in these parameters at any of the compound of Formula II doses tested. FIG. 11 shows that TT701 causes no change in Hematocrit with the doses tested. Examination of vital sign data from clinical studies of TT701 did not yield any significant changes in systolic blood pressure, diastolic blood pressure, or heart rate. Subjects in Study GPBC received visual acuity, visual field assessment, and fundoscopy examinations as well as an Ocular Surface Disease Index test before and after receiving TT701. There were no clinically relevant changes in these assessments.

### Example 12: Anabolic Effects of the Compound of Formula II

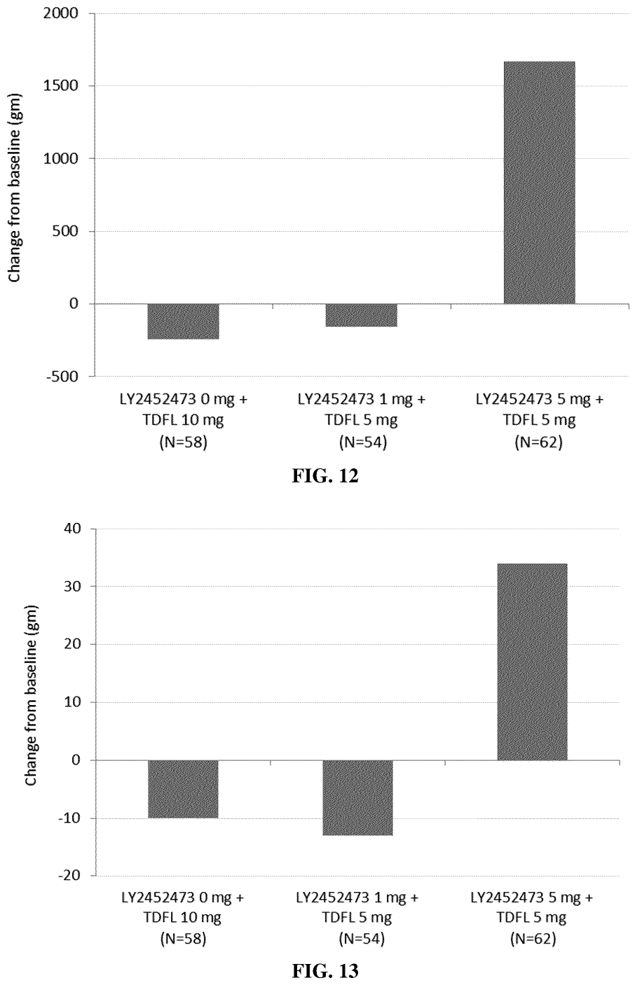

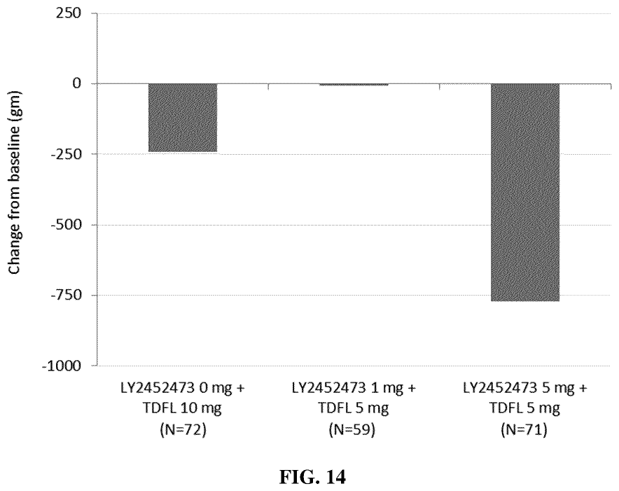

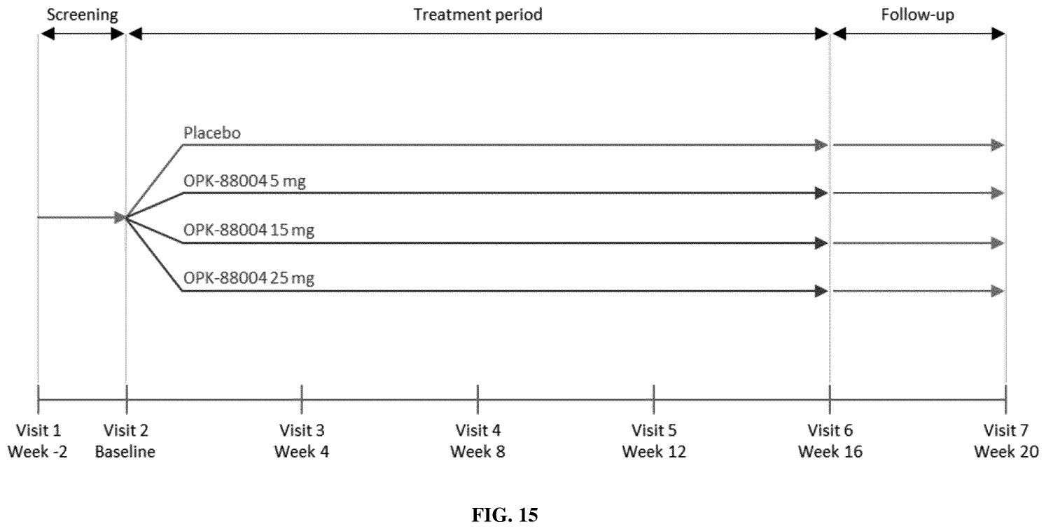

The available data indicate that the compound of Formula II may have had the agonist effects of an androgen via decreased fat mass and increased LBM. In a multiple dose study (Study GPBC), healthy subjects exposed to the compound of Formula II for 28 days demonstrated clinically and statistically significant increases in LBM and calf area (by CT). The Phase 2 Study for ED also included exploratory measures for lower extremity muscle strength and power, LBM and fat mass. In this study, 12 weeks of daily treatment with the compound of Formula II indicated that the compound of Formula II may have had the agonist effects of an androgen via decreased fat mass and increased LBM. Patients receiving a combination treatment of 5 mg the compound of Formula II+5 mg tadalafil had a reduction of fat body mass and an increase (improvement) of LBM compared with patients receiving 10 mg tadalafil. Lower extremity muscle power, as measured by the stair climb, was increased (improved) in patients receiving a combination treatment of 5 mg the compound of Formula II+5 mg tadalafil compared with patients receiving 10 mg tadalafil. The results are illustrated in FIGS. 12, 13, and 14.

FIG. 11 illustrates the fat Mass mean change from baseline to week 12: Study GPEC.

### Example 13: Clinical Study for Treating Muscle Wasting, Low Muscle Strength, or Low Physical Function in ESRD Patients on Dialysis

Patients will include male ESRD patients on dialysis with testosterone levels of less than 300 ng/dL. The male patients will also be 50 years or older. Patients will also include post-menopausal female ESRD patients on dialysis who are 50 years or older. The study will consist of 20 male patients and 20 female patients per treatment arm.

Patients will also receive, in separate arms, doses of the SARM of Formula II (OPK-88004, 5 mgs) versus placebo. The doses will be administered post dialysis on dialysis days. The patients will be on hemodialysis.

The treatment will last for 16 weeks. One month of screening will occur before the 16 week treatment and one month of follow up will occur once the 16 week treatment ends.

Both studies referenced above may also include an arm having patients treated with both the SARM and a controlled release calcifediol dosage form. The dosage amount and frequency of dosing will depend upon the particular patients in the study(ies) but will most likely be once to three times a week of 300 to 900 μg 25(OH)D3 in a suitable oral dosage form such as a wax matrix or other controlled release formulation in a soft gel capsule or hard shell capsule.

The primary endpoint of the study will be lean body mass (by DXA) or, in a combination study, a combination of lean body mass increase and the treatment of vitamin D insufficiency along with a lowering of iPTH of about 30 percent. The second endpoints will include muscle strength, physical function, fat mass, bone content/biomarkers, quality of life (energy levels, mood, sexual function), safety, and/or pharmacokinetics.

### Example 14: Clinical Study Administering the SARM of Formula II (TT701/OPK88004) in Patients with Lower Urinary Tract Symptoms (LUTS) Secondary to BPH and Measuring LBM and PSA

The clinical study treated 114 male subjects (aged: 64.6 years; baseline testosterone: 447.3 ng/dL) with lower urinary tract symptoms (LUTS) secondary to BPH by administering TT701 and evaluated the change in lean body mass (“LBM”), serum PSA levels, and the safety of TT701 as compared to placebo.

Treatment with OPK-88004 at 15 mg and 25 mg resulted in decreased fat mass at 16 weeks vs placebo (15 mg: −1.47 kg, p=0.0002; 25 mg: −1.49 kg, p=0.0003) and increased LBM (15 mg: 1.61 kg, p=0.001; 25 mg: 1.96 kg, p=0.0004), demonstrating androgen agonistic effects on body composition (Table 12). A total of 74 subjects completed the study (28 in the placebo group, 23 in the 15 mg group, and 23 in the 25 mg group) and 40 subjects were terminated early. The primary endpoint, PSA at 16 weeks did not change with 15 mg OPK-88004, and significantly decreased (p=0.006, mITT) in the 25 mg OPK-88004 group (Table 13).

TABLE 12
Change from baseline in lean body mass
(LBM) and fat mass at week 16.

| | Change in Lean | Change in |
|---|---|---|
| | Body Mass (kg) | Fat mass (kg) |
| | (week 16) | (week 16) |
| OPK-88004 15 mg | 1.61 kg | −1.47 kg |
| (N = 22) | (p = 0.001) | (p = 0.0002) |
| OPK-88008 25 mg | 1.96 kg | −1.49 kg |
| (N = 35) | (p = 0.0004) | (p = 0.0003) |

TABLE 13
Change from baseline in prostate specific antigen
(PSA) at week 16.

| | Change in Prostate Specific |
|---|---|
| | Antigen (week 16 to baseline) |
| OPK-88004 15 mg | 1.48 (p = 0.79) |
| OPK-88008 25 mg | −15.44 (p = 0.006) |
| Placebo | −2.96 (p = 0.57) |

TABLE 14
Change from baseline in LBM and fat mass at week 12 stratified
by baseline levels of testosterone (<340 ng/dL or ≥ 340 ng/dL)

| | Change in Lean | | Change in | |
|---|---|---|---|---|
| | Body Mass (%) | | Fat Mass (%) | |
| | (week 12) | | (week 12) | |
| | Testosterone (ng/dL) | | | |
| | <340 ng/dL | ≥340 ng/dL | <340 ng/dL | ≥340 ng/dL |
| | N = 32 | N = 20 | N = 32 | N = 20 |
| OPK-88004 | 1.60% | 0.88% | −1.85% | −0.95% |
| 5 mg (N = 52) | | | | |

### Example 16: Concentrations and Pharmacokinetic Parameters for Different Dosing Regimens of OPK-88004

The human pharmacokinetics (PK) of OPK-88004 was investigated following single and repeated doses. Following tₘₐₓ, OPK-88004 concentrations appear to decline in a biexponential fashion characterized by a short distribution phase and a long terminal phase. Exposure appears to increase approximately dose proportionally in the clinical dosing range. Steady state was achieved within 28 days of dosing. Accumulation of up to 2.5-fold was observed with multiple dosing. The disposition and PK profile of OPK-88004 may exhibit important safety and efficacy advantages in patients with significantly compromised renal function such as ESRD patients on dialysis. These therapeutic benefits of more controlled exposure may result in improved LBM and physical/muscle strength preservation, physical function improvement and muscle wasting prevention. OPK-88004 bioexponential PK profile is important for the optimization of side effect benefit/risk ratio in special populations including dialysis patients.

OPK-88004 has shown significantly improved LBM in healthy males with BPH and ED. In healthy females, the most significant testosterone decreases are noted between the ages 21-40 with a 50% decrease (approx. 20 ng/dL) by 40 years of age (Zumoff B, Strain G W, Miller L K, Rosner W. Twenty-four-hour mean plasma testosterone concentration declines with age in normal premenopausal women. J Clin Endocrinol Metab 80: 1429-1430). Physical frailty in older females is 2 times more prevalent than in males (Ruan, Qingwei, et al. “Sexual dimorphism of frailty and cognitive impairment: Potential underlying mechanisms.” Molecular medicine reports 16.3 (2017): 3023-3033.). Therefore, it is likely that OPK-88004 will exhibit higher efficacy in females including improved LBM that will translate into better physical strength/function and muscle strength vs males.

Previous phase 2 trial in aging males with ED demonstrated that a 5 mg dose improved lean body mass (1.67 kg) of OPK-88004 over 12 weeks (See Example 15) with similar potency to the higher doses of 15 mg (1.57 kg) and 25 mg (1.92 kg) of OPK-88004 that were tested over 16 weeks in aging males with BPH (See Example 14). The biexponential PK profile resulting in accumulation of drug at lower doses such as 5 mg over a 5-6-week period results in increased exposure and PK profile of OPK-88004 leading to increased LBM and physical function at lower doses in the absence of safety issues arising from higher doses. In indications such as muscle wasting in patients on dialysis whose physiologic responses are highly compromised due to CKD and dialysis, it would be expected that patients respond favorably and benefit from the PK properties of OPK-88004 at these low doses.

TABLE 15
Change in Lean Body Mass from Baseline (kg)

| GPEC (week 12) | SAR-202 (week 16) | |
|---|---|---|
| OPK-88004 | OPK-88004 | OPK-88004 |
| 5 mg (N = 72) | 15 mg (N = 22) | 25 mg (N = 19) |
| 1.70 kg (p < 0.001) | 1.61 kg (p = 0.0001) | 1.96 kg (p = 0.0004) |

OPK-88004 has been shown to increase bone mineral content and bone density, as well as increase LBM, physical strength and function. These two findings combined suggest the patients suffering from muscle wasting, sarcopenia or frailty would benefit from treatment with OPK-88004 and potentially have a reduction of the incidence of falls and bone fractures. Combining OPK-88004 treatment with Vitamin D3 analogues such as calcifediol (Rayaldee), could lead to further and significant improvements in bone formation and physical function resulting in reduced incidence of falls and fractures.

From this novel biexponential OPK-88004 data, it is anticipated that lower doses of OPK-88004 such as 5 mg will be safe in dialysis patients with ESRD, yet achieve LBM necessary to improve physical strength and function. The biexponential PK properties of OPK-88004 should permit once daily dosing of lower doses in dialysis patients with ESRD with an optimal safety and efficacy profile including improvement of muscle wasting and physical strength/function, and frailty.

### Example 17: Simulations of OPK-88004 Once Daily for 14 Days

Simulations of doses between 5 mg and 25 mg once daily are displayed below in FIG. 4. The different shaded areas are the 20%, 50% and 80% prediction interval and the solid line represents the typical subject prediction. The dashed red vertical line represents the moment the steady state is achieved

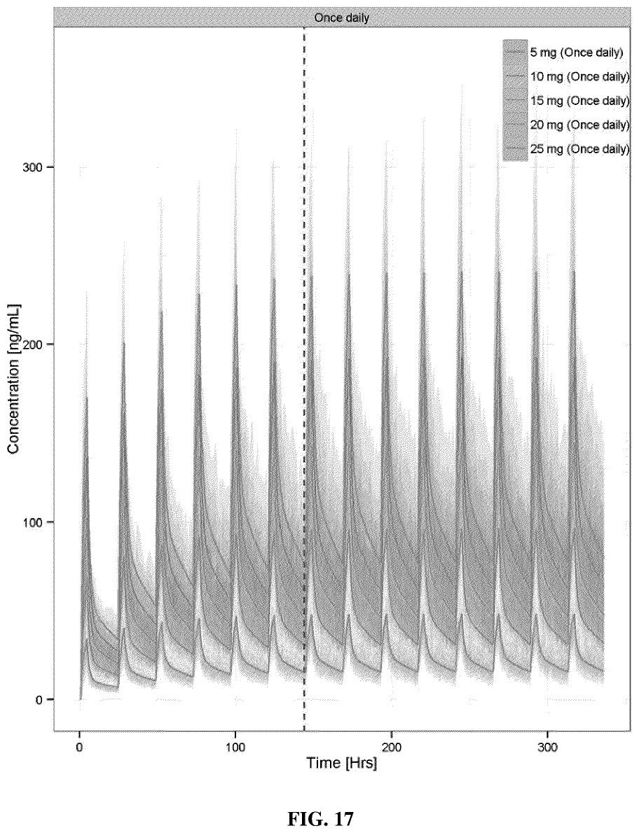

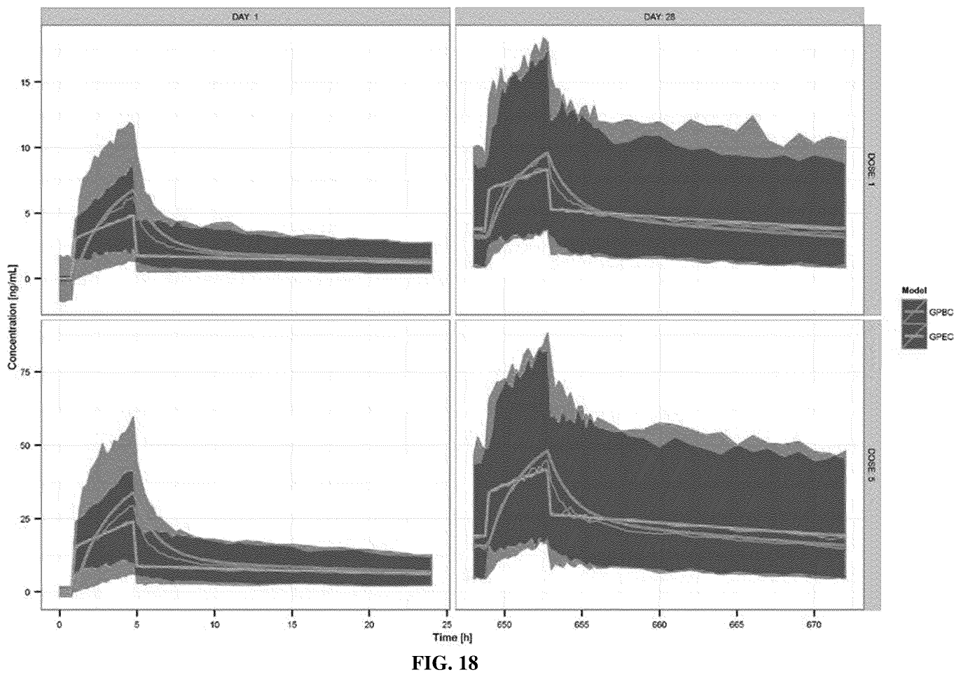

FIG. 17 shows a simulation of concentrations profile of OPK-88004 for once daily administration for 14 days.

The population predicted profiles at day 14 were used to calculate the steady state pharmacokinetic parameters (Cmax, Cavg, Cmin, and AUC0-τ) with a non-compartmental method and are presented in Table 5 below. The steady state of OPK-88004 is achieved at Day 6.

TABLE 16
Steady State Pharmacokinetic Parameters for
Different Dosage of OPK-88004 Once daily

| | Cmax | Cavg | Cmin | AUC0 − τ |
|---|---|---|---|---|
| Dosing Regimen | (ng/mL) | (ng/mL) | (ng/mL) | (h*ng/mL) |
| mg (Once daily) | 48.17 | 23.98 | 15.55 | 575.48 |
| 10 mg (Once daily) | 96.33 | 47.96 | 31.11 | 1150.95 |
| 15 mg (Once daily) | 144.50 | 71.93 | 46.66 | 1726.43 |
| 20 mg (Once daily) | 192.66 | 95.91 | 62.21 | 2301.90 |
| mg (Once daily) | 240.83 | 119.89 | 77.76 | 2877.39 |

0 − τ = dosing interval of 0-24 h

Based on the 100 replications of the 15 mg pharmacokinetic profile, the five greatest simulated Cmax were 386.18, 386.61, 400.37, 428.15, and 457.52 ng/mL.

Based on the 100 replications of the 25 mg pharmacokinetic profile, the five greatest simulated Cmax were 735.11, 737.76, 742.86, 762.54, and 816.27 ng/mL

### Example 18: Simulations of OPK-88004 Compared in GPBC Study and GPEC Study

FIG. 18 shows stimulated profiles for two studies referred to in the previous Examples, the GPBC Study and the GPEC Study. When two simulated profiles were compared, the simulated shapes were different; however, the main PK characteristics including Cmax for 5%, 50% and 95% of subjects (depicted as 3 separate lines in red or blue for each study) and variability were comparable.

Simulations were performed assuming a once a day dosing for 28 days (1 and 5 mg), and a full PK visit on days 1 and 28.

In FIG. 18, the bold solid line were the PK profile of the typical subject, the shaded area were the 90% prediction interval, and the solid lines were the 5%, 50%, and 95% percentiles. The median (50% percentile) was well in accordance with the typical subject.

The main PK characteristics (in terms of Cmax, AUC, elimination, and accumulation), as well as the variability, were found to be comparable. The simulated comparison supports 5 mg dosing to day 28 in the two different studies.

1. A method of treating (I) symptoms or conditions associated with renal disease in a subject in need of treatment thereof, (II) symptoms or conditions associated with renal disease in a subject, wherein said symptom or condition is selected from at least one of muscle wasting, low muscle strength, low testosterone, or low physical function, (III) end stage renal disease or treating at least one of muscle wasting, low muscle strength, or low physical function in a subject having end stage renal disease, (IV) treating symptoms as a result of secondary hypogonadism induced by renal replacement therapy or kidney failure in a subject having renal disease, (V) treating symptoms as a result of secondary hypogonadism induced by renal replacement therapy or kidney failure in a subject having renal disease, wherein said symptoms are the loss in bone mass, bone strength, muscle mass, or muscle strength, comprising administering a therapeutically effect amount of at least one tetrahydrocyclopenta[b]indole compound to said subject, wherein the tetrahydrocyclopenta[b]indole compound has Formula I: 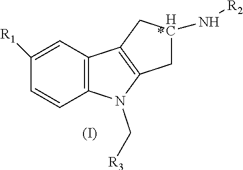
wherein the C* atom may be R, S or R/S configuration;
R₁ represents cyano, —CH═NOCH₃, —OCHF₂, or —OCF₃;
R₂ represents —COR₂ₐ or —SO₂R_{2b};
R₂ₐ represents (C₁-C₄)alkyl, (C₁-C₄)alkoxy, cyclopropyl, or —NRaRb;
R_{2b} represents (C₁-C₄)alkyl, cyclopropyl, or —NRaRb;
Ra and Rb each independently is H or (C₁-C₄)alkyl; and
R₃ represents a heteroaryl group selected from the group consisting of pyridinyl, pyrimidinyl, pyrazinyl, pyridazinyl, thiazoloy isothiazolyl, and thiadiazolyl, each optionally substituted with 1 or 2 substituents independently selected from the group consisting of methyl, ethyl, bromo, chloro, fluoro, —CHF₂, —CF₃, hydroxyl, amino and —NHCH₂CO₂H, or a pharmaceutically acceptable salt thereof.

2. (canceled)

3. The method of claim 1, wherein the subject has stage 3, 4 or 5 chronic kidney disease.

4. (canceled)

5. (canceled)

6. The method of claim 1, wherein R₁ is CN, —CH═NOCH₃ or —OCF₃.

7. The method of claim 1, wherein (I) R₂ is —COR₂ₐ or —SO₂R_{2b} wherein R₂ₐ is (C₁-C₄)alkyl, (C₁-C₄)alkoxy, cyclopropyl, or —N(CH₃)₂ and R_{2b} is (C₁-C₄)alkyl, cyclopropyl, —N(CH₃)₂ or —N(C₂H₅)₂; (II) R₂ is —COR₂ₐ or —SO₂R_{2b} and R₂ₐ is ethyl, isopropyl, methoxy, ethoxy, propoxy, isopropoxy, isobutoxy, tert-butoxy, cyclopropyl, or —N(CH₃)₂, and R_{2b} is methyl, ethyl, propyl, cyclopropyl, —N(CH₃)₂ or —N(C₂H₅)₂; (III) R₂ is —COR₂ₐ and R₂ₐ is selected from ethyl, isopropyl, methoxy, ethoxy, propoxy, isopropoxy, isobutoxy, tert-butoxy, cyclopropyl, or —N(CH₃)₂; (IV) R₂ is —SO₂R_{2b}, and R_{2b} is methyl, ethyl, propyl, cyclopropyl, —N(CH₃)₂ or —N(C₂H₅)₂; (V) R₂ is —COR₂ₐ and R₂ₐ is selected from ethyl, isopropyl, methoxy, ethoxy, propoxy, isopropoxy, isobutoxy, tert-butoxy, cyclopropyl, or —N(CH₃)₂; or (VI) R₂ is —COR₂ₐ and the “C*” carbon center is in the S configuration; or R₂ is —SO₂R_{2b} and the “C*” carbon center is in the R configuration.

8. (canceled)

9. (canceled)

10. (canceled)

11. (canceled)

12. (canceled)

13. The method of claim 1, wherein (I) R₃ is a heteroaryl group selected from the group consisting of pyridinyl, pyrimidinyl, pyrazinyl, pyridazinyl, thiazolyl, isothiazolyl, and thiadiazolyl, each optionally substituted with one or more substituents independently selected from the group consisting of methyl, bromo, chloro, fluoro, —CHF₂, hydroxyl, amino and —NHCH₂CH₂CO₂H; (II) R₃ represents 6-fluoro-pyridin-2-yl, pyridine-2-yl, 3-hydroxy-pyridin-2-yl, 6-difluoromethyl-pyridin-2-yl, 2-amino-pyridin-3-yl, 2-carboxymethylamino-pyridin-3-yl, pyrimidin-4-yl, pyrimindin-2-yl, 2-chloro-pyrimidin-4-yl, thiazol-4-yl, 2-methyl-thiazol-4-yl, 2-chloro-thiazol-4-yl, thiazol-2-yl, thiazol-5-yl, thiazol-5-yl, 4-amino-thiazol-5-yl, pyrazine-2-yl, 5-methyl-pyrazin-2-yl, 3-chloro-pyrazin-2-yl, pyridazin-3-yl, 5-bromo-isothiazol-3-yl, isothiazol-3-yl, 4,5-dichloro-isothiazol-3-yl, or [1,2,5]thiadiazol-3-yl; or (III) R₃ is pyridine-2-yl, 2-amino-pyridin-3-yl, thiazol-5-yl, or 4-amino-thiazol-5-yl.

14. (canceled)

15. (canceled)

16. (canceled)

17. The method of claim 1, wherein (I) said tetrahydrocyclopenta[b]indole compound is carbamic acid, N-[(2S)-7-cyano-1,2,3,4-tetrahydro-4-(2-pyridinylmethyl)cyclopent[b]indol-2-yl]-, 1-methylethyl ester; or (II) said tetrahydrocyclopenta[b]indole compound has the structure of formula II:

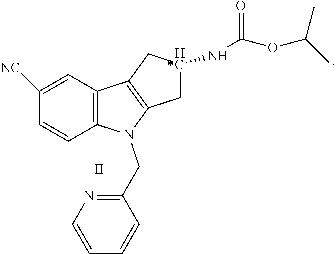

18. A method of treating (I) end stage renal disease, (II) at least one of muscle wasting, low muscle strength, or low physical function in a subject having end stage renal disease, (III) symptoms as a result of secondary hypogonadism induced by renal replacement therapy or dialysis, or (IV) kidney failure in a subject having end stage renal disease, comprising administering a therapeutically effect amount of the compound of Formula II or a pharmaceutically acceptable salt thereof to said subject, wherein the compound has Formula II:

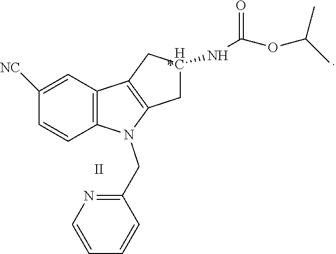

19. (canceled)

20. The method of claim 18, wherein the compound of formula II is in the (2S) configuration.

21. The method according claim 18, where said subject has chronic kidney disease (“CKD”), stage five CKD, end-stage renal disease (“ESRD”), kidney failure, is undergoing dialysis.

22. (canceled)

23. (canceled)

24. (canceled)

25. (canceled)

26. (canceled)

27. The method of claim 18, wherein said dialysis is hemodialysis or peritoneal dialysis.

28. The method of claim 18, wherein said subject is male, said subject is male and has low testosterone, said subject is male with a low testosterone level less than 300 nanograms per deciliter (ng/dL), said subject is female, or said subject is female and is post-menopausal.

29. (canceled)

30. (canceled)

31. (canceled)

32. (canceled)

33. The method of claim 28, wherein said subject is 50 years or older or said subject is between 50 and 80 years old.

34. (canceled)

35. The method claim 18, further comprising administering a therapeutically effect amount of a second composition (“second compound”) to said subject, wherein said second compound is administered simultaneously to said tetrahydrocyclopenta[b]indole compound.

36. (canceled)

37. The method of claim 35, wherein said second compound comprises (I) drugs typically provided to the ESRD patients, (II) vitamins and minerals improving renal function, (III) carotenoids, vitamin C, vitamin D, vitamin E, vitamin K, vitamin B1, vitamin B2, vitamin B3, vitamin B5, vitamin B6, vitamin B7, vitamin B9, or vitamin B12, (IV) selenium or zinc, (V) antioxidant amino acids, (VI) L-cysteine or glutathione, (VII) extended-release calcifediol (ERC), (VIII) an ERC comprising (3β,5Z,7E)-9,10-secocholesta-5,7,10(19)-triene-3,25-diol monohydrate, (IX) sexual function drugs, (X) a sexual function drug comprising tadalafil, (XI) vitamin D pro-hormone, (XII) a vitamin D pro-hormone comprising 25(OH)D3 or 25(OH)D2, or (XIII) calcitriol or a vitamin D analog.

38. (canceled)

39. (canceled)

40. (canceled)

41. (canceled)

42. (canceled)

43. (canceled)

44. (canceled)

45. The method of claim 37, wherein said ERC comprises the following structure: 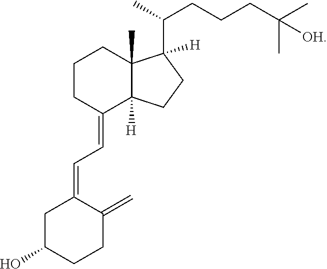 [SMILES: C=C1CC[C@H](O)C/C1=C/C=C1\CCC[C@]2(C)[C@@H]([C@H](C)CCCC(C)(C)O)CC[C@@H]12]
and which is further combined with a controlled release excipient.

46. (canceled)

47. (canceled)

48. (canceled)

49. (canceled)

50. (canceled)

51. (canceled)

52. The method of claim 37, wherein said first compound is present about 0.5 mg to about 50 mg or and wherein said second compound is present in an amount of about 1 to about 1000 μg.

53. The method of claim 37, wherein said first compound is present about 5 mg or and wherein said second compound is present in an amount of about 30 μg.

54. The method of claim 37, wherein either compound or both compounds are present in an amount of about 5 mg.

55. The method of claim 52, wherein either compound or both compounds are administered once daily, either compound or both compounds are administered orally, or either compound or both compounds are administered in a gelatin capsule.

56. The method of claim 55, where said administration occurs after a dialysis treatment.

57. The method of claim 52, wherein either compound or both compounds are in a single dosage form, each compound is in a single dosage form and co-administered, or both compounds in a single dosage form and administered in a single dosage form.

58. (canceled)

59. (canceled)

60. The method of claim 57, wherein said single dosage form is a modified-release dosage or wherein said single dosage form is a modified-release dosage comprising an extended-release core of said second compound and an immediate-release coating of said tetrahydrocyclopenta[b]indole compound.

61. (canceled)

62. (canceled)

63. (canceled)

64. The method of claim 37, wherein said treatment improves muscle wasting, said treatment improves physical strength or physical function, or said treatment reduces sarcopenia or frailty.

65. (canceled)

66. (canceled)

67. The method claim 37, wherein the administration results in a change compared to a baseline prior to the initiation of treatment in any one of lean body mass (LBM), physical function, muscle power, muscle strength, physical strength, fat mass, bone mineral content, bone content, bone biomarkers, fatigue, quality of life parameters, sexual function, safety, or pharmacokinetics.

68. (canceled)

69. The method of claim 67, wherein said change is an increase in LBM comprising at least 1 kg or wherein said quality of life parameters include any one of energy levels, mood, or sexual function.

70. (canceled)

71. (canceled)

72. (canceled)

73. (canceled)

74. The method of claim 67, wherein said change is measured after 16 weeks of administration or is measured after 12 or 16 weeks of treatment.

75. A pharmaceutical composition comprising a compound of formula I or formula II and an extended release dosage form of calcifediol and acceptable excipients in a fixed unit combination oral dosage form.

76. The pharmaceutical composition according to claim 75, wherein said fixed unit combination dosage form is a capsule or a tablet and the compound of formula II is carbamic acid, N-[(2S)-7-cyano-1,2,3,4-tetrahydro-4-(2-pyridinylmethyl)cyclopent[b]indol-2-yl]-, 1-methylethyl ester.

77. (canceled)
